(19) United States
(12) Patent Application Publication
(10) Pub. No.: US 2010/0093618 A1
(43) Pub. Date: Apr. 15, 2010

(54) POLYPEPTIDE CAPABLE OF INHIBITING HIV-1 TRANSCRIPTION AND REPLICATION AND USES THEREOF

(76) Inventors: Ileana QUINTO, Catanzaro (IT); Giuseppe Scala, Catanzaro (IT)

Correspondence Address: Steinfl & Bruno, 301 N Lake Ave Ste 810, Pasadena, CA, 91101, US

(21) Appl. No.: 12/252,279

(22) Filed: Oct. 15, 2008

(51) Int. Cl. A61K 38/16; C07K 14/00; A61P 31/18

(52) U.S. Cl. 514/12; 530/350

(57) ABSTRACT

A polypeptide inhibitor of transcription and replication of Human Immunodeficiency Virus (HIV-1), and related compositions and methods.

### CROSS REFERENCE

The present disclosure is related to Applicants' paper, “IκB-α Represses the Transcriptional Activity of the HIV-1 Tat Transactivator by Promoting Its Nuclear Export “by Antimina Puca, Giuseppe Fiume, Camillo Palmieri, Francesca Trimboli, Francesco Olimpico, Giuseppe Scala, and Ileana Quinto in Journal of Biological Chemistry vol. 282, no. 51, pp. 37146-37157, Dec. 21, 2007, herein incorporated by reference in its entirety.

### FIELD

The present disclosure relates to the field of virology, more particularly to the use of a polypeptide inhibitor to inhibit transcription and/or replication of HIV, and to treat Acquired Immune Deficiency Syndrome (AIDS) and/or related conditions.

### BACKGROUND

The Acquired Immune Deficiency Syndrome (AIDS) is caused by the retrovirus HIV-1 and is now a pandemic especially in the underdeveloped areas, such as the Africa continent, where about 40 million people are infected with human immunodeficiency virus, type 1 (HIV-1). The development of vaccines and therapies aimed at eradicating HIV-1 is a primary goal in the struggle against AIDS.

Current AIDS therapies are based on the use of combinations of viral reverse transcriptase and protease inhibitors. This kind of therapy is designated as “Highly Active Antiretroviral Therapy” (HAART). HAART has proven to be effective for controlling the progression of AIDS, but it is ineffective for the eradication of the viral tissue reservoirs. On the other hand, discontinuing HAART results in a rapid increase of viremia and progression of the infection.

Thus, if on one hand uninterrupted administration of the HAART therapy is mandatory in AIDS patients, this treatment, on the other hand, results in high costs and in the onset of severe side effects due to modifications in the fat metabolism, such as increased cholesterol and triglycerides blood levels, lipodistrophy and heart circulatory and renal function's alterations. A further problem that in certain cases can be associated with HAART is the appearance of therapy-resistant viral strains, such as HAART-resistant HIV-1 strains.

Virus-cell membrane fusion inhibitors as well as virus integrase inhibitors have been developed recently and tested in clinical trials. However, the appearance of HIV-1 mutant strains resistant to these kinds of inhibitors has been observed in certain cell cultures.

### SUMMARY

Provided herein, are polypeptides that can be used as inhibitor of transcription and replication processes of HIV-1. In particular, provided herein are polypeptides that are derivative of inhibitor κB-α (IκB-α) and related methods and compositions.

According to a first aspect, a polypeptide is disclosed that is an inhibitor of HIV-1 transcription and replication. The polypeptide comprises: a nuclear localization signal of IκB-α, or a derivative thereof; a C-terminal nuclear export signal of IκB-α, or a derivative thereof; and a binding site of IκB-α for an HIV-1 Tat transactivator, or a derivative thereof.

According to a second aspect, a polypeptide is disclosed that is an inhibitor of HIV-1 transcription and replication. The polypeptide comprises: an amino acid sequence consisting of SEQ ID NO:12, or a derivative thereof; an amino acid sequence consisting of SEQ ID NO:13, or a derivative thereof; and/or an amino acid sequence consisting of SEQ ID NO:14, or a derivative thereof.

According to a third aspect, a method of inhibiting HIV-1 transcription and replication in a host cell is disclosed. The method comprises administering to said host cell an effective amount of a polypeptide inhibitor herein disclosed.

According to a fourth aspect a composition for inhibiting HIV-1 transcription and replication in a host cell, is disclosed. The composition comprises a polypeptide inhibitor herein disclosed and a compatible vehicle.

According to a fifth aspect, a method of treating or preventing a condition associated with presence in an individual of HIV-1 is disclosed. The method comprises administering to the individual a therapeutic effective amount of a polypeptide inhibitor herein disclosed.

According to a sixth aspect, a pharmaceutical composition for inhibiting HIV-1 transcription and replication is disclosed. The pharmaceutical composition comprises the polypeptide inhibitor herein disclosed and a pharmaceutically acceptable vehicle.

According to a seventh aspect, a method for producing a polypeptide inhibitor of HIV-1 transcription and replication is disclosed. The method comprises: selecting a nuclear localization signal of IκB-α, or a derivative thereof, thus obtaining a selected nuclear localization signal; selecting a C-terminal nuclear export signal of IκB-α, or a derivative thereof, thus obtaining a selected nuclear export signal; and selecting a binding site of IκB-α for an HIV-1 Tat transactivator, or a derivative thereof, thus obtaining a selected Tat binding site. The method also comprises forming said polypeptide inhibitor of HIV-1 transcription and replication with the selected nuclear localization signal, the selected nuclear export signal and the selected Tat binding site.

The polypeptides, methods and compositions herein disclosed allow, in certain embodiments, inhibition of HIV-1 virus and related activity other than the reverse transcriptase and protease activities.

The polypeptides, methods and compositions herein disclosed further allow, in certain embodiments, to be used in connection new antiviral medicaments capable of controlling HAART-resistant HIV-1 strains.

The polypeptides, methods and compositions herein disclosed further allow, in certain embodiments, to be used as antiviral medicaments acting on alternative steps of the HIV-1 life cycle than the ones currently targeted by medicaments of the art.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description, serve to explain the principles and implementations of the disclosure.

FIG. 1 shows inhibition of HIV-1 transcription according to some embodiments herein described. Panel A shows a diagram illustrating the results of HeLa cells transfection performed with pLTRluc wild type or deleted of the NF-κB or Sp1 sites. Panels B to D show results of HeLa cells transfection with pLTRluc wild type (B) or deleted of NF-κB (C) or Sp1 (D) sites (0.5 μg) in presence or absence of p3XFLAG-CMV-Tat and pCMV4-HA-IκB-α. In panels A to D, the mean values±S.E. (n=4) are shown. For NF-κB-deleted LTR, the asterisks indicate a statistically significant inhibition according to Student's t test (*, p=0.01; **, p=0.002; ***, p=0.0007).

FIG. 2 shows inhibition of viral expression and replication according to some embodiments herein disclosed. Panel A shows a diagram illustrating the results of p50^{-/-} p65^{-/-} MEFs cell transfection with the NF-κB-deleted pLTRluc, with or without p3XFLAG-CMV-Tat, pRc/CMV-HA-hCycT1, and pCMV4-HA-IκB-α. The mean values±S.E. (n=4) are shown. The asterisks indicate a statistically significant inhibition according to Student's t test (without hCycT1: *, p=0.008; **, p=0.0009; ***, p=0.0002; with hCycT1: *, p=0.006; **, p=0.001; ***, p=0.0001). Panel B shows a diagram illustrating the results of Jurkat cells electroporation with pCMV4-HA-IκB-α or empty vector, IκB-α siRNA, or control siRNA and infected with VSV-G-pseudotyped NL4-3.Luc.R-E-virions that carry the wild type LTR (left panel) or the NF-κB-deleted LTR (right panel). Virus production was monitored by measuring the luciferase activity in cell extracts 48 h post-infection (top). The expression level of IκB-α was detected in cell extracts by Western blotting with anti-IκB-α C-15 (bottom). Panel C shows a schematic representation of the viral genome of NL-IκB-M and NL-IκB-as carrying the wild type LTR or NL-ΔκB-IκB-M and NL-ΔκB-IκB-as carrying the NF-κB-deleted LTR. Panel D shows a diagram illustrating the results of Jurkat cells infection performed with equal amounts of the wild type LTR viruses, NL-IκB-M and NL-IκB-as (left panel), or NF-κB-deleted LTR viruses, NL-ΔκB-IκB-M and NL-ΔκB-IκB-as (right panel).

FIG. 3 shows viral inhibition by various polypeptides according to some embodiments herein disclosed. Panel A shows a schematic representation of wild type IκB-α. Panel B shows a diagram illustrating the results of p50^{-/-} p65^{-/-} MEFs transfection with the NF-κB-deleted LTRluc in presence or absence of p3XFLAG-CMV-Tat, pRc/CMV-HA-hCycT1, and pCMV4-HA-IκB-α 1-317 or the indicated IκB-α mutants. The mean values±S.E. (n=7) are shown. The asterisks indicate a statistically significant inhibition according to Student's t test (IκB-α 1-317, p=0.0017; IκB-α 1-287, p=0.0001; IκB-α 1-280, p=0.004; IκB-α 72-317, p=0.0036; IκB-α 72-287, p=0.0007). Panel C shows results of p50^{-/-} p65^{-/-} MEFs transfection with p3× FLAG-CMV-Tat and pCMV4-HA-IκB-α mutants as shown in Panel B analyzed by Western blotting (WB) for the expression of transfected genes.

FIG. 4 shows binding of a polypeptide herein disclosed to a viral transactivator according to some embodiments herein described. Panel A, shows results of HeLa cells, MEFs and p50^{-/-} p65^{-/-} MEFs transfection with pCMV4-HA-IκB-α, GST pull-down and related Western blotting (WB) of protein complexes with anti-HA and anti-GST antibodies. Panel B, shows results of incubation of HeLa cell extracts with GST-Tat or GST, GST pulldown, and related Western blotting (WB) of protein complexes with anti-IκB-α (C-15) and anti-GST antibodies. Panel C, shows results of HeLa cells transfection with pCMV4-HA-IκB-α, subsequent incubation of cell extracts with GST-Tat or GST, GST pulldown, and related Western blotting (WB) of protein complexes with anti-HA and anti-GST antibodies. Panel D shows a schematic representation of wild type Tat and the mutants Tat C(22,25,27)A and Tat R(49-57)A. Panel E shows results of HeLa cells transfection with p3XFLAG-CMV-Tat, p3XFLAG-CMV-Tat R(49-57)A, or p3XFLAG-CMV-Tat C(22,25,27)A, subsequent incubation of cell extracts with GST-Tat or GST, GST pulldown, and related Western blotting (WB) of protein complexes with anti-FLAG and anti-GST antibodies. Panel F, shows results of HeLa cells transfection with p3XFLAG-CMV-Tat, p3XFLAG-CMV-Tat R(49-57)A, or p3XFLAG-CMV-Tat C(22,25,27)A in presence or absence of pCMV4-HA-IκB-α.

FIG. 5 shows a structure-function relationship of residues of polypeptides herein disclosed according to some embodiments herein disclosed. Panel A shows a schematic representation of IκB-α proteins used for the GST-Tat pulldown in some embodiments herein disclosed. Panel B shows incubation of [³⁵S]methionine-labeled IκB-α proteins with GST-Tat or GST, GST pulldown, and related autoradiography analysis (top panels) and Western blotting with anti-GST antibody (bottom panels).

FIG. 6 shows nuclear export promotion of viral proteins by s herein disclosed according to some embodiments herein disclosed. Panel A shows HeLa cell transfection with p3XFLAG-CMV-Tat in presence or absence of pCMV4-HA-IκB-α 1-317, pCMV4-HA-IκB-α 120-317, pCMV4-HA-IκB-α 1-269, pCMV4-HA-IκB-α 72-287, pCMV4-HA-IκB-α 72-269, or pCMV4-HA-IκB-α 72-287 L(272,274,277)A. The scale bar is 10 μm. Panel B shows Western blotting (WB) analysis of cell extracts from HeLa cells transfected as illustrated in panel A with anti-HA, anti-FLAG, and anti-γ-tubulin antibodies.

FIG. 7 shows fluorescence-based image analysis of a viral protein and a polypeptide inhibitor herein described according to some embodiments herein described. In the panels, each point represents the values of a single cell; the solid diagonal line indicates equal nuclear and cytoplasmic fluorescence (nuclear/cytoplasmic fluorescence ratio=1/1); the upper and lower dashed lines indicate, respectively, 10/1 and 1/10 nuclear/cytoplasmic fluorescence ratios.

FIG. 8 shows a nuclear export activity and inhibition of viral protein by a polypeptide herein disclosed according to some embodiments herein disclosed. Panel A shows p50^{-/-} p65^{-/-} MEFs cell transfection with p3XFLAG-CMV-Tat and pCMV4-HA-IκB-α 1-317 N/C NES, analyzed by confocal microscopy. The scale bar is 10 μm. Panel B shows the fluorescence-based analysis of FLAG-Tat and HA-IκB-α. Panel C shows results of p50^{-/-} p65^{-/-} MEFs cell transfection with the NF-κB-deleted LTRluc in presence or absence of p3XFLAG-CMV-Tat, pRc/CMV-HA-hCycT1, and pCMV4-HA-IκB-α 1-317 or pCMV4-HA-IκB-α N/C NES. The mean values±S.E. (n=4) are shown. The asterisk indicates a statistically significant inhibition according to Student's t test (p=0.0017). Panel D shows results of incubating [³⁵S]methionine-labeled IκB-α wild type and IκB-α N/C NES with GST-Tat or GST, GST pulldown, and related autoradiography analysis (top panels) and Western blotting with anti-GST antibody (bottom panel).

FIG. 9 is a schematic model of viral inhibition by polypeptides herein described according to some embodiments herein described. Panel A shows summary of inhibition, binding and nuclear export of Tat exhibited by relevant IκB-α mutants. Panel B shows a schematic representation of the mechanism of Tat inhibition by IκB-α.

FIG. 10 shows that lack of apoptosis induction in cell transfected with a polypeptide herein disclosed according to some embodiments of the present disclosure. A diagram illustrates results of HeLa cells transfection with or without p3XFLAG-CMV-Tat and pCMV4-HA-IκB-α and Western blot analysis of cell extracts with anti-Caspase-3, cleaved PARP, anti-HA, anti-FLAG and anti-γ-tubulin antibodies. Caspase-3 and PARP cleavaged indicates the controls.

FIG. 11 shows viral integration in cells treated with polypeptide inhibitors according to some embodiments herein disclosed. Diagrams illustrate viral integration in Jurkat cells electroporated with pCMV4-HA-IκB-α or empty vector, IκB-α siRNA or control siRNA (500 pmol), and infected with the wild type (left panel) or NF-κB-deleted (right panel) VSV-Gpseudotyped NL4-3.Luc.R-E-.

FIG. 12 shows structure-function relationship in polypeptide inhibitors according to some embodiments herein disclosed. A diagram illustrated results of p50^{-/-} p65^{-/-} MEFs (3×10⁵) transfection with the NF-κB-deleted LTRluc in presence or absence of p3XFLAG-CMV-Tat, pRc/CMVHA-hCycT1 and pCMV4-HA-IκB-α 1-317 or the mutants IκB-α 120-317, IκB-α 72-269, IκB-α 72-287L(272, 274, 277)A at the indicated doses. The mean values±SEM (n=3) are shown.

FIG. 13 shows structure-function relationship in polypeptide inhibitors according to some embodiments herein disclosed. Panels A and B show CLUSTALW-based multiple sequence alignment of the sixth ankyrin of IκB-α with the other five ankyrins of IκB-α (A), and the ankyrins of the human IκB family (B). Numbers refer to the amino acid sequence of the proteins. Dark gray boxes indicate identities, light gray boxes indicate conservative changes. The TRIQQQL sequence of IκB-α that binds Tat is boxed. Panel C show results of incubation of in vitro translated p105 and FLAG-p100 with GST-Tat or GST, GST pull-down, and related Western blotting analysis with anti-p105 (top, lanes 1-3), anti-FLAG (top, lanes 4-6) and anti-GST antibody (bottom).

FIG. 14 shows inhibition of viral proteins by polypeptide inhibitors according to some embodiments herein disclosed in presence of a nuclear export inhibitor. A diagram illustrates p50^{-/-} p65^{-/-} MEFs transfection with the NF-κB-deleted pLTRluc in presence or absence of p3XFLAG-CMV-Tat, pRc/CMV-HA-hCycT 1 and pCMV4-HA-IκB-α 1-317. The mean values±SEM (n=3) are shown. The asterisk indicates a statistically significant inhibition according to the Student's t-test (P=0.001) (top). Cellular extracts (25 μg) were analyzed by Western blotting with anti-IμB-α (C-15), anti-FLAG and anti-γ-Tubulin antibodies (bottom).

### DETAILED DESCRIPTION

Compounds, compositions and related methods and uses are herein provided to interfere and in particular inhibit HIV virus and various conditions associated with said virus. In particular, a polypeptide is disclosed that is capable of inhibiting HIV-1 transcription and replication processes.

The term “polypeptide” as used herein indicates an organic polymer composed of two or more amino acid monomers and/or analogs thereof. The term “polypeptide” includes amino acid polymers of any length including full length proteins and peptides, as well as analogs and fragments thereof. As used herein the term “amino acid”, “amino acidic monomer”, or “amino acid residue” refers to any of the twenty naturally occurring amino acids including synthetic amino acids with unnatural side chains and including both D L optical isomers. The term “amino acid analog” refers to an amino acid in which one or more individual atoms have been replaced, either with a different atom, isotope, or with a different functional group but is otherwise identical to its natural amino acid analog. The term “protein” as used herein indicates a polypeptide with a particular secondary and tertiary structure that in certain cases can interact with other biomolecules including other proteins, DNA, RNA, lipids, metabolites, and small molecules. The term “fragment” as used herein with reference to a polypeptide or a protein, indicates a first polymer that constitutes a portion of a second polymer with the first polymer detachable from the second polymer by enzymatic, chemical or other reactions which are identifiable by a skilled person.

The terms “inhibiting” and “inhibit”, as used herein indicate the activity of decreasing a biological reaction or process, which include but are not limited to polynucleotide transcription, polynucleotide replication and replication of a biological system, such as an organism (e.g. animal, plant, fungus, or micro-organism) or an infective agent (e.g. a virus). Accordingly, the term “inhibitor” as used herein indicates a substance capable of decreasing a certain biological reaction or process, and includes but is not limited to, any substance that decreases said biological reaction or process by reducing or suppressing the activity of another substance (e.g. an enzyme) associated to the biological reaction or process to be inhibited, e.g. by binding, (in some cases specifically), said other substance. Inhibition of the biological reaction or process can be detected by detection of an analyte associated with the biological reaction or process. The term “detect” or “detection” as used herein indicates the determination of the existence, presence or fact of an analyte or related signal in a limited portion of space, including but not limited to a sample, a reaction mixture, a molecular complex and a substrate. A detection is “quantitative” when it refers, relates to, or involves the measurement of quantity or amount of the analyte or related signal (also referred as quantitation), which includes but is not limited to any analysis designed to determine the amounts or proportions of the analyte or related signal. A detection is “qualitative” when it refers, relates to, or involves identification of a quality or kind of the analyte or related signal in terms of relative abundance to another analyte or related signal, which is not quantified.

The term “HIV” as used herein indicates a lentivirus (a member of the retrovirus family) that can lead to acquired immunodeficiency syndrome (AIDS), a condition in humans in which the immune system begins to fail, leading to life-threatening opportunistic infections. The term “condition” as used herein indicates a physical status of the body of an individual (as a whole or of one or more of its parts), that does not conform to a standard physical status associated with a state of complete physical, mental and social well-being for the individual. Conditions herein described include but are not limited disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms. In particular, HIV viruses include HIV-1 virus a lentivirus (species Human immunodeficiency virus 1) that is the most prevalent HIV virus and is also called HTLV-III.

The terms “transcription” and “transcription process” as used herein indicate a process of transcribing a polynucleotide sequence information (and in particular DNA sequence information) into RNA sequence information, which is typically but not necessarily associated with the process of constructing a messenger RNA molecule using a DNA molecule as a template with resulting transfer of genetic information to the messenger RNA in a biological system. Accordingly the terms “transcription” and “transcription process” when referred to HIV indicate the process of transcribing sequence information from the viral genome into RNA sequence information a host cell and includes but not limited to the process of constructing a messenger RNA from a double stranded DNA formed in the host cell upon entry of the virus into the host cell.

The terms “replication” and “replication process” as used herein indicate the act or process of reproducing or duplicating a polynucleotide sequence information or a biological system, and include but is not limited to the process of copying a single or double stranded DNA or RNA molecule to form one or more corresponding molecules, and to the process by which a certain organism or infective agent reproduces multiple copies of itself. Accordingly, the terms “replication” and “replication process” as used herein with reference to HIV indicate the act or process of reproducing the virus into a host cell.

Inhibitor polypeptides herein disclosed consists of or are derivatives of inhibitor κB-α (IκB-α) one of the best characterized and ubiquitous member of the IκB family, that contains six ankyrins, a nuclear localization signal (NLS), and two nuclear export signals located at the amino, terminus (N-NES) and carboxyl terminus (C-NES), all identifiable by a skilled person upon reading of the present disclosure. The term “derivative” as used herein with reference to a first polypeptide (e.g., IκB-α), indicates a second polypeptide that is structurally related to the first polypeptide and is derivable from the first polypeptide by a modification that introduces a feature that is not present in the first polypeptide while retaining functional properties of the first polypeptide. Accordingly, a derivative polypeptide of IκB-α, or of any portion thereof, e.g. NLS or C-NES, usually differs from the original polypeptide or portion thereof by modification of the amino acidic sequence that might or might not be associated with an additional function not present in the original polypeptide or portion thereof. A derivative polypeptide of IκB-α, or of any portion thereof retains however one or more functional activities that are herein described in connection with IκB-α or portion thereof in association with the inhibiting activity of IκB-α.

In some embodiments, the polypeptide inhibitor herein described comprises the nuclear localization signal (NLS), the C-terminal nuclear export signal (C-NES) and the Tat binding site of the inhibitor κB-α (IκB-α) amino acid sequence.

The wording “nuclear localization signal” as used herein indicate an amino acid sequence in which acts like a ‘tag’ on the exposed surface of a protein, and is used to target the protein to the cell nucleus through the Nuclear Pore Complex and/or to direct a newly synthesized protein into the nucleus via its recognition by cytosolic nuclear transport receptors. Typically, this signal consists of one or more short sequences of positively charged lysines or arginines. Accordingly, the NLS of IκB-α indicates the amino acid sequence used to target IκB-α to the cell nucleus identifiable by a skilled person upon reading of the present disclosure.

The wording “nuclear export signal” as used herein indicates a short amino acid sequence (typically of 5-6 hydrophobic residues) in a protein that targets it for export from the cell nucleus to the cytoplasm through the nuclear pore complex. Typically an NES is recognized and bound by exportins. Accordingly, the C-terminal nuclear export signal of IκB-α is the amino acid sequence located on the C-terminal portion of IκB-α that is used for export from the cell nucleus to the cytoplasm identifiable by a skilled person upon reading of the present disclosure.

The wording “Tat binding site” as used herein indicates a region on a molecule form a chemical bond with a viral transactivator Tat. In particular, the Tat binding site of IκB-α is the region of IκB-α able to specifically bind a Tat transactivator of HIV.

The wording “specific” “specifically” or specificity” as used herein with reference to the binding of a molecule to another refers to the recognition, contact and formation of a stable complex between the molecule and the another, together with substantially less to no recognition, contact and formation of a stable complex between each of the molecule and the another with other molecules. Exemplary specific bindings are antibody-antigen interaction, cellular receptor-ligand interactions, polynucleotide hybridization, enzyme substrate interactions etc.

In some embodiments, the polypeptide inhibitor comprises or consists of the amino acid positions 72 to 287 of IκB-α. In some embodiments, the polypeptide inhibitor comprises the amino acid positions 110 to 120 of IκB-α. In some embodiments, the polypeptide inhibitor comprises the amino acid positions 265 to 277 of IκB-α. In some embodiments, the polypeptide inhibitor comprises the amino acid positions 263 to 269 of IκB-α.

In some embodiments, the polypeptide inhibitor comprises or consists of the amino acid sequence designated as SEQ ID NO:1, SEQ ID NO: 12, SEQ ID NO:13 and/or SEQ ID NO: 14 in the sequence listing, or an amino acid sequence that is at least 90% identical to SEQ ID NO:1, SEQ ID NO: 12, SEQ ID NO:13 and/or SEQ ID NO: 14. Further embodiments comprises polypeptide inhibitors having identity percentages of at least 91%, at least 92%, at least 93%, at least 94%, at least 95%, at least 96%, at least 97%, at least 98% or at least 99% with a polypeptide having SEQ ID NO:1, SEQ ID NO: 12, SEQ ID NO:13 and/or SEQ ID NO: 14.

A polypeptide has a certain percent “sequence identity” to another polypeptide, meaning that, when aligned, that percentage of amino acids is the same when comparing the two sequences.

In particular, percentage identity can be determined by aligning two sequences to be compared, determining the number of identical residues in the aligned portion, dividing that number by the total number of residues in the sequence that provides the basis for comparison, and by multiplying the result by 100. A percentage identity can also be determined with reference to a specified region of a polypeptide against another polypeptide or region thereof.

In particular, to determine sequence identity, sequences can be aligned using methods and computer programs identifiable by a skilled person. “Sequence alignment” indicates the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA [9, 10]. When using all of these programs, the preferred settings are those that results in the highest sequence similarity.

For example, the “identity” or “percent identity” with respect to a particular pair of aligned amino acid sequences can refer to the percent amino acid sequence identity that is obtained by ClustalW analysis (version W 1.8 available from European Bioinformatics Institute, Cambridge, UK), counting the number of identical matches in the alignment and dividing such number of identical matches by the greater of (i) the length of the aligned sequences, and (ii) 96, and using the following default ClustalW parameters to achieve slow/accurate pairwise alignments—Gap Open Penalty: 10; Gap Extension Penalty: 0.10; Protein weight matrix: Gonnet series; DNA weight matrix: IUB; Toggle Slow/Fast pairwise alignments=SLOW or FULL Alignment. Other techniques for alignment are described in various publications (see e.g. [11]). Of particular interest are alignment programs that permit gaps in the sequence, such as the Smith-Watennan [12] and the GAP program using the Needleman and Wunsch alignment method [13].

In some embodiments, the percentage identity can be determined following optimal alignment between the polypeptides sequences to be compared. Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well known in the art and described, [14, 15]. The BLOSUM62 matrix is often used as a default scoring substitution matrix in sequence alignment protocols such as Gapped BLAST 2.0. The gap existence penalty is imposed for the introduction of a single amino acid gap in one of the aligned sequences, and the gap extension penalty is imposed for each additional empty amino acid position inserted into an already opened gap.

The alignment is defined by the amino acids positions of each sequence at which the alignment begins and ends, and optionally by the insertion of a gap or multiple gaps in one or both sequences so as to arrive at the highest possible score. While optimal alignment and scoring can be accomplished manually, the process is facilitated by the use of a computer-implemented alignment algorithm, e.g., gapped BLAST 2.0, described in (14), and made available to the public at the National Center for Biotechnology Information (NCBI) Website (www.ncbi.nlm.nih.gov). Optimal alignments, including multiple alignments, can be prepared using, e.g., PSI-BLAST, available through the NCBI website and described in [14]. “Sequence similarity” takes into account (a) the functional impact of amino acid substitutions, (b) amino acid insertions and deletions and (c) the length and structural complexity of a sequence. A “sequence similarity score” is determined by means of a sequence alignment as described above. The “protein similarity score” “S” is a value calculated based on scoring matrix and gap penalty. The higher the score, the more significant the alignment, and the higher the degree of similarity between the queried sequences.

The polypeptides inhibitors herein disclosed can be either isolated from a natural source or synthesized by chemical or biochemical methods as well as by way of recombinant microorganism technologies, all identifiable by a skilled person. Thus, those methods and technologies will not be further described herein in detail.

Provided herein is also a method of inhibiting transcription and replication of HIV-1 in a host cell. The method comprises administering to the host cell an effective amount of a polypeptide inhibitor herein disclosed. In some embodiments, inhibiting transcription and replication of HIV-1 is performed in vitro. In some embodiments, inhibiting transcription and replication of HIV-1 is performed in vivo.

An effective amount of the polypeptide of the disclosure can be within the range of between about 10 nM and 1 mM and more particularly from about 100 nM to about 100 μM.

In methods herein disclosed, inhibiting transcription and replication of HIV-1 is performed by a polypeptide inhibitor herein disclosed by directly inhibiting the transcriptional activity of the HIV-1 Tat transactivator, which is carried out independently of the Nuclear Factor-κB (NF-κB). In particular, directly inhibiting of the transcriptional activity of the HIV-1 Tat transactivator is performed by the polypeptides inhibitors herein disclosed by direct binding to the Tat transactivator. In some embodiments, the binding of the polypeptide to Tat results in the nuclear export and cytoplasmatic sequestration of the viral transactivator and in the inhibition of the transcription and replication of HIV-1.

In other embodiments, a method of treating or preventing a condition associated with presence in an individual of HIV-1 virus. The method comprises administering to the individual a therapeutically effective amount of the polypeptide inhibitor herein disclosed. The term “individual” as used herein includes a single biological organism including but not limited to animals and in particular higher animals and in particular vertebrates such as mammals and in particular human beings. Conditions associated with presence of HIV-1 virus include but are not limited to Acquired immune deficiency syndrome or acquired immunodeficiency syndrome (AIDS or Aids) a set of symptoms and infections resulting from the damage to the human immune system caused by the human immunodeficiency virus (HIV) that progressively reduces the effectiveness of the immune system and leaves individuals susceptible to opportunistic infections and tumors.

The term “treatment” as used herein indicates any activity that is part of a medical care for or deals with a condition medically or surgically.

The term “prevention” as used herein indicates any activity which reduces the burden of mortality or morbidity from a condition in an individual. This takes place at primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

A therapeutically effective amount of the polypeptide of the disclosure can be within the range of between about 10 nM and 1 mM and more particularly from about 100 nM to about 100 μM.

In some embodiments, the polypeptides herein disclosed are comprised in a composition together with a suitable vehicle. The term “vehicle” as used herein indicates any of various media acting usually as solvents, carriers, binders or diluents for the polypeptide or polypeptides that are comprised in the composition as an active ingredient.

In some embodiments, where the composition is to be administered to an individual the composition can be a pharmaceutical composition for inhibiting HIV-1 transcription and replication, and comprises the polypeptide inhibitor of the disclosure and a pharmaceutically acceptable vehicle.

In some embodiments, the polypeptides herein disclosed are included in pharmaceutical compositions together with an excipient or diluent. In particular, in some embodiments, pharmaceutical compositions are disclosed which contain at least a peptide as described above, in combination with one or more compatible and pharmaceutically acceptable vehicle, and in particular with pharmaceutically acceptable diluents or excipients.

The term “excipient” as used herein indicates an inactive substance used as a carrier for the active ingredients of a medication. Suitable excipients for the pharmaceutical compositions herein disclosed include any substance that enhances the ability of the body of an individual to absorb the peptides or combinations thereof. Suitable excipients also include any substance that can be used to bulk up formulations with the peptides or combinations thereof, to allow for convenient and accurate dosage. In addition to their use in the single-dosage quantity, excipients can be used in the manufacturing process to aid in the handling of the peptides or combinations thereof concerned. Depending on the route of administration, and form of medication, different excipients may be used. Exemplary excipients include but are not limited to antiadherents binders coatings disintegrants, fillers, flavors (such as sweeteners) and colors, glidants, lubricants, preservatives, sorbents.

The term “diluent” as used herein indicates a diluting agent which is issued to dilute or carry an active ingredient of a composition. Suitable diluent include any substance that can decrease the viscosity of a medicinal preparation.

In certain embodiments, compositions and, in particular, pharmaceutical compositions can be formulated for parenteral administration. Exemplary compositions for parenteral administration include but are not limited to sterile aqueous solutions, injectable solutions or suspensions including the polypeptide inhibitor herein disclosed. In some embodiments, a composition for parenteral administration can be prepared at the time of use by dissolving a powdered composition, previously prepared in lyophilized form, in a biologically compatible aqueous liquid (distilled water, physiological solution or other aqueous solution).

In certain embodiments, compositions and, in particular, pharmaceutical compositions can be formulated for systemic administration. Exemplary compositions for systemic administration include but are not limited to a tablet, a capsule, drops, and suppositories.

The Examples section of the present disclosure illustrates examples of the polypeptides and related compositions and methods herein described as well as the studies carried out by applicants in order to investigate the functional and physical interactions of IκB-α with the HIV-1 Tat transactivator, which is indispensable for viral replication. The experimental evidences obtained by Applicants led to the identification of the IκB-α sequence required for Tat binding and inhibition (SEQ ID NO:1) and elucidated the underlying mechanism of action, involving binding of IκB-α to Tat and nuclear export of the viral transactivator to the cell cytoplasm.

Further advantages and characteristics of the present disclosure will become more apparent hereinafter from the following detailed disclosure in the Examples give by way or illustration only with reference to an experimental section.

### EXAMPLES

The compounds compositions methods and systems herein disclosed are further illustrated in the following examples, which are provided by way of illustration and are not intended to be limiting.

The experiments described in the examples are performed using the following experimental procedures.

### Experimental Procedures

### Plasmids

pLTRluc contains the U3 and R regions of the pNL4-3 molecular clone of HIV-1 upstream of the luciferase gene [1]. pSV-β-gal was purchased from Promega. To generate p3XFLAG-CMV-Tat, p3XFLAG-CMV-Tat C(22,25,27)A, and p3XFLAG-CMV-Tat R(49-57)A, the sequence of Tat was amplified from the pGEX-2T-Tat expressing vectors (2) and ligated to EcoRI/XbaI-digested p3XFLAG-CMV-7.1 (Sigma). pRc/CMV-HA-IκB-α S32/36A was previously described [3]. The plasmids expressing the IκB-α mutants 120-317, 1-280, 1-269, 1-242, 72-287, and 72-269 were generated by PCR-mediated amplification of the IκB-α sequence from pCMV4-HA-IκB-α with appropriate forward and reverse primers followed by ligation to the HindIII/XbaI-digested pCMV4-HA.

The mutant IκB-α 72-287 L(272,274,277)A was generated by site-directed mutagenesis of the IκB-α 72-287 sequence using the forward primer ATACAGCAGCAGCTGGGCCAGGCGACAGCAGAAAACGCGCAGATGCTGCCA GAGA (SEQ ID NO:2) and the reverse primer CTGGCCCAGCTGCTGCTGTATCCGGGTGCTTGGGCGGCC (SEQ ID NO:3), with the mutated triplets indicated in bold type. The mutant IκB-α N/C NES was generated by site-directed mutagenesis of IκB-α 1-317 at the level of the N-NES with the forward primer AAGGAGCTGCAG GAG GCG CGC GCG GAG CCG CAG GAG GTG (SEQ ID NO:4) and the reverse primer CTCCTGCAGCTCCTTGACCATGGAGTCCA (SEQ ID NO:5), and at the level of C-NES with the same primers described for IκB-α 72-287 L(272,274,277)A. The mutated nucleotides are shown in bold type.

The pGEX-2T-IκB-α plasmids were generated by PCR-mediated amplification of the IκB-α sequences from the plasmid pCMV4-HA-IκB-α followed by ligation to BamHI/EcoRI-digested pGEX-2T (Amersham Biosciences). The pcDNA3 plasmids expressing the IκB-α mutants under the T7 promoter were generated by PCR amplification of the IκB-α genes from the pCMV4-HA-IκB-α plasmids followed by ligation to KpnI/XbaI-digested pcDNA3 (Invitrogen). All of the constructs were verified by automated DNA sequencing. To generate the NF-κB-deleted pNL4-3.Luc.R-E-, the XhoI/HindIII fragment of pNL4-3.Luc.R-E, which contains the 3′ LTR, was replaced with the corresponding fragment from the NF-κB-deleted pLTRluc.

To generate pNL-ΔκB-IκB-M and pNL-ΔκB-IκB-as, the viral plasmids pNL-IκB-M and pNL-IκB-as (1) were digested with NaeI to isolate the 2.35- and 1.61-kb fragments, which contain the viral sequence from the unique NaeI site within the IκB-αS32/36A-FLAG insert in the nef region in sense or antisense orientation, respectively, to the unique NaeI site of pNL4-3 (10,346 nucleotides) in the flanking region downstream to the 3′ LTR. The DNA fragments were ligated to the NaeI site of pBlueScript K+ (Stratagene) to generate pBSK-IκB-M and pBSK-IκB-as, respectively.

The two tandem κB sites within the 3′ LTR of pBSK-IκB-M and pBSK-IκB-as were deleted by site-directed mutagenesis using the forward primer FNLDKB CGAGCTTGCTACAAGGGATCTAGATCCAGGGAGGCGTGGCCTGGGC (SEQ ID NO:6) and the reverse primer RNLDKB TCCTTGTAGCAAGCTCGATGTCAGCAGTTCTTGAAGTAC (SEQ ID NO:7) to generate pBSK-ΔκB-IκB-M and pBSK-ΔκB-IκB-as, respectively. The mutated sequence of κB sites is shown in bold type in the forward primer. The viral plasmids pNL-ΔκB-IκB-M and pNL-ΔκB-IκB-as were generated by replacement of the NaeI-digested 2.35- and 1.61-kb DNA fragments with the corresponding region from pBSK-ΔκB-IκB-M and pBSK-ΔκB-IκB-as, respectively.

### Cell Culture, Transfection, and Luciferase Assay

HeLa and MEFs were cultured in Dulbecco's modified Eagle's medium (Invitrogen), Jurkat cells in RPMI (Invitrogen). The culture media were supplemented with 10% heat-inactivated fetal calf serum and 2 mM L-glutamine at 5% CO₂ and 37° C. The cells were transfected with DNA by using FuGENE 6 (Roche Applied Science), and the total amounts of DNA were equalized by transfection of pRc/CMV empty vector (Invitrogen). For luciferase assays, pSV-β-gal plasmid (0.2 μg) was co-transfected with the pLTRluc plasmids to monitor the transfection efficiency. Forty-eight hours post-transfection, the cells were lysed in lysis buffer of Dual Light Luciferase System (Tropix, Bedford, Mass.). The luciferase and β-galactosidase activities were evaluated by using the Dual Light luciferase system (Tropix, Bedford, Mass.) in a bioluminometer (Turner Biosystem, Sunnyvale, Calif.). The ratio of firefly luciferase activity to β-galactosidase activity was expressed as relative light units.

### Pseudotyped Virions and Single-Round Infection

293-T cells were transfected with wild type or NF-κB-deleted pNL4-3.Luc.R⁻E⁻ (10 μg) and pVSV.G (10 μg) expressing the G protein of the vescicular stomatitis virus. Forty-eight hours post-transfection, the cell supernatants were collected, and the virions were measured by p24 enzyme-linked immunosorbent assay. Jurkat cells (4×10⁶) were transfected by electroporation with pCMV4-HA-IκB-α or empty vector (30 μg) or with IκB-α siRNA or control siRNA (500 pmol) (Dharmacon, Lafayette, CO) and 48 h later were infected with VSV-Luc virions (500 ng of p24) by spinoculation [4]. The luciferase activity was measured in cell extracts 48 h post-infection.

### Viral Integration

Genomic DNA was extracted from aliquots of infected cells (2×10⁶) using TRIzol (Invitrogen) and amplified with primers that annealed in the U5 region of the LTR (MH 531) and in the 5′ end of the gag gene (MH 532). The reaction mixture (25 μl) contained genomic DNA (200 ng), primers (600 nM), and 1× iQ SYBR Green Supermix (Bio-Rad). Real time PCR was performed by using iCycler Apparatus (Bio-Rad). After an initial denaturation step (95° C. for 8 min), the cycling profile for total HIV-1 DNA was 50 cycles consisting of 95° C. for 10 s, 60° C. for 10 s, and 72° C. for 6 s.

Viral DNA was normalized to cellular genomic glyceraldehyde-3-phosphate dehydrogenase. Primers were as follows: MH531, TGTGTGCCCGTCTGTTGTGT (SEQ ID NO:8); MH532, GAGTCCTGCGTCGAGAGAGC (SEQ ID NO:9); glyceraldehyde-3-phosphate dehydrogenase forward, GAAGGTGAAGGTCGGAGTC (SEQ ID NO:10); and glyceraldehyde-3-phosphate dehydrogenase reverse, GAAGATGGTGATGGGATTTC (SEQ ID NO:11). The HIV-1 DNA copy number was measured as reported [5].

### Viral Stocks and Cell Culture Infection

293-T cells were transfected with viral plasmids, and the viral production was measured by p24 enzyme-linked immunosorbent assay. Jurkat cells (5×10⁴ cells) were infected with p24 (0.3 ng) of viral stocks, and the cell supernatants were collected every 3 days for p24 assay. Equal volumes of fresh medium were replaced into the cultures at the same time.

### Statistical Analysis

The data were reported as the means±S.E. and the statistical significance of differences between means was assessed by using the two-tail unpaired Student's t test. The differences between the means were accepted as statistically significant at the 95% level (p=0.05).

### Cell Extracts and Western Blotting

Cells (5×10⁶) were harvested, washed in cold PBS, and lysed on ice in 500 μl of lysis buffer containing 50 mM Tris-HCl, pH 8.0, 150 mM NaCl, 1% sodium deoxycholate, 0.2% SDS, 2 mM EDTA, 1% Triton X-100, 5 mM DTT, 1× protease inhibitor mixture EDTA-free (Roche Applied Science). After centrifugation at 15,000×g for 15 min at 4° C., the supernatant was collected, and aliquots of proteins were resuspended in loading buffer (125 mM Tris-HCl, pH 6.8, 5% SDS, 1% bromphenol blue, 10% β-mercaptoethanol, 25% glycerol), resolved on 10% SDS-PAGE, transferred to polyvinylidene difluoride membrane (Millipore, Bedford, Mass.), and incubated with primary antibodies (1:1000) followed by incubation with horseradish-peroxidase-linked mouse or rabbit IgG (1:2000) (Amersham Biosciences) in PBS containing 5% nonfat dry milk (Bio-Rad).

The proteins were detected by chemiluminescence using the Amersham Biosciences ECL system. The primary antibodies were as follows: anti-HA (F7), anti-GST (B-14), anti-IκB-α (C-15), and normal mouse serum from Santa Cruz Biotechnology (Santa Cruz, Calif.); anti-FLAG M2 and anti-γ-tubulin from Sigma-Aldrich; anti-caspase-3 and cleaved poly(ADP-ribose)polymerase (Asp214) antibody from Cell Signaling Technology, Inc. (Danvers, Mass.).

### GST Pulldown

GST fusion proteins were produced in Escherichia coli strain BL21 as previously described [2]. Bacterial cultures (500 ml) were grown to exponential phase and induced with 0.25 mM isopropyl-β-D-thiogalactopyranoside (Sigma-Aldrich) for 3 h to express GST fusion proteins. The bacteria were lysed by sonication in buffer A (1× PBS, 1 mM DTT, 1 mM phenylmethylsulfonyl fluoride, and 1×protease inhibitor mixture EDTA-free), and the lysate was clarified by centrifugation at 27,000×g for 30 min at 4° C. The supernatant was incubated with 1 ml of a 50% (v/v) slurry of glutathione-Sepharose beads (Amersham Biosciences) previously equilibrated in buffer A.

After incubation on a rotating wheel at 4° C. for 2 h, the beads were washed five times with buffer A and subjected to a high salt wash (0.8 M NaCl) to free the fusion proteins from contaminating bacterial nucleic acids [6]. GST fusion proteins were eluted with 500 μl of 50 mM Tris-HCl containing 10 mM glutathione and 1 mM DTT. The eluted GST fusion proteins were dialyzed against dialysis buffer (1× PBS, 1 mM DTT, 10% glycerol), and aliquots (5-10 μg) were conjugated with glutathione-Sepharose (20 μl) in 500 μl of binding buffer (50 mM Tris-HCl, pH 8.0, 150 mM NaCl, 1% sodium deoxycholate, 0.2% SDS, 2 mM EDTA, 3% Triton X-100, 5 mM DTT, 1× protease inhibitor mixture EDTA-free).

The GST fusion proteins conjugated with glutathione-Sepharose were collected by centrifugation at 700×g for 5 min at 4° C., and aliquots (5-10 μg) were incubated with cell extracts (200 μg) in 500 μl of binding buffer supplemented with 1 μg/μl of bovine serum albumin on a rocking platform for 3 h at 4° C. To remove nucleic acids, the cell extracts were treated with micrococcal nuclease (0.2 unit/μl) for 30 min at 28° C. Protein complexes were collected by centrifugation at 700×g for 5 min at 4° C., washed in binding buffer, and resuspended in loading buffer (125 mM Tris-HCl, pH 6.8, 5% SDS, 1% bromphenol blue, 10% β-mercaptoethanol, 25% glycerol).

The proteins were resolved on 10% SDS-PAGE, transferred to polyvinylidene difluoride membranes, and analyzed by immunoblotting with the indicated antibodies. The pcDNA3 plasmids expressing the IκB genes under the T7 promoter were used as templates to translate in vitro the [³⁵S]methionine-labeled IκB proteins by using the TNT Quick Coupled transcription/translation systems (Promega). Aliquots (10 μl) of translation mixture were incubated with GST-Tat or GST proteins (10 μg) in 500 μl of binding buffer supplemented with 1 μg/μl of bovine serum albumin on a rocking platform for 3 h at room temperature. Following GST pulldown, the proteins were separated by 12% SDS-PAGE and analyzed by autoradiography and immunoblotting with antibodies.

### Co-Immunoprecipitation

Cell extracts were performed in PBS containing 1% Triton X-100 and 1× Protease Inhibitor Mixture EDTA-free. Antibodies (2.5 μg) were preincubated with protein G-Sepharose (Amersham Biosciences) (20 μl) in 50 μl of immunoprecipitation buffer (PBS containing 2% Triton X-100, 300 mM NaCl, 5 mM DTT, 1× Protease Inhibitor Mixture EDTA-free) overnight at 4° C. on a rocking platform. The protein G-Sepharose-coupled antibodies were incubated with cell extract (500 μg) in 500 μl of immunoprecipitation buffer overnight at 4° C. on a rocking platform. The immunocomplexes were collected by centrifugation at 700×g for 5 min at 4° C., washed in immunoprecipitation buffer, and resuspended in SDS gel loading buffer. The proteins were separated on 10% SDS-polyacrylamide gel, transferred to polyvinylidene difluoride membrane, and analyzed by immunoblotting with antibodies.

### Confocal Microscopy

Confocal microscopy was performed as previously described [7]. HeLa cells were seeded on poly-L-lysine-treated glass coverslips, fixed, and permeabilized using Cytofix/Cyto-Perm kit (BD Biosciences Pharmingen, San Diego, Calif.). To visualize FLAG-Tat and HA-IκB-α, the immunostaining was performed with anti-FLAG-M2-FITC mAb (F-4049; Sigma-Aldrich) and anti-HA rabbit antiserum (SC-805; Santa Cruz Biotechnologies) followed by goat anti-rabbit Alexa Fluor568 (A11011; Molecular Probes, Eugene, Oreg.). The nuclei were stained with TO-PRO-3 iodide (T3605; Molecular Probes).

The coverslips were mounted on glass slides by using ProLong Antifade Kit (P7481; Molecular Probes). The images were collected on a Leica TCS-SP2 confocal microscope (Leica Mycrosystems, Wetzlar, Germany) with a 63x Apo PLA oil immersion objective (NA 1.4) and 60-μm aperture. Z stacks of images were collected using a step increment of 0.2 μm between planes. FLAG-Tat was visualized by excitation with an argon laser at 488 nm and photomultiplier tube voltage of 420 mV. HA-IκB-α was detected using a krypton laser at 568 nm and photomultiplier tube voltage of 650 mV. The nuclei were detected using a krypton laser at 613 nm and photomultiplier tube voltage of 450 mV. Single optical sections using 4× averaging were acquired by sequential scanning to collect the images in three channels. For quantitative analysis of the nuclear and cytoplasmic protein levels, horizontal sections scanned through the nucleus and cytoplasm of thirty representative cells were evaluated.

Fluorescence-based assessment of protein levels was performed by image analysis using the LEICA Scan TCS-SP2 software (Leica Mycrosystems). Quantization was performed on 8-bit gray scale images with no saturated pixels. The mean nuclear or cytoplasmic fluorescence was measured as the ratio between total fluorescence/total pixels at the nuclear or cytoplasmic level in individual cells. The relative nuclear or cytoplasmic fluorescence was calculated as the ratio between the mean nuclear or cytoplasmic fluorescence and the mean fluorescence of the whole cell.

### Example 1

### Inhibition of Tat-Mediated Transactivation and Replication of HIV-1 Independently of NF-κB Activity

To determine the effect of IκB-α on the transcriptional activity of Tat, HeLa cells were transiently transfected with the luciferase gene under the control of the wild type or NF-κB- or Sp1-deleted HIV-1 LTR in the presence or absence of Tat and IκB-α.

In particular, in a first series of experiments HeLa cells (3×10⁵) were transfected with pLTRluc wild type or deleted of the NF-κB or Sp1 sites (0.5 μg). NF-κB nuclear factor κB) and Sp1 are cellular transcription factors which, together with the viral transactivator Tat, are known to regulate HIV-1 transcription through interaction with the viral Long Terminal Repeats (LTRs). The Luciferase activity was measured 48 h post-transfection.

In a second series of experiments, HeLa cells (3×10⁵) were transfected with pLTRluc wild type or deleted of NF-κB or Sp1 sites (0.5 μg) in presence or absence of p3XFLAG-CMV-Tat (0.5 μg) and pCMV4-HA-IκB-α (0.5, 1, and 2 μg). The luciferase activity was measured 48 h post-transfection. Fold activation was calculated relative to transfection in the absence of Tat and IκB-α expression plasmids.

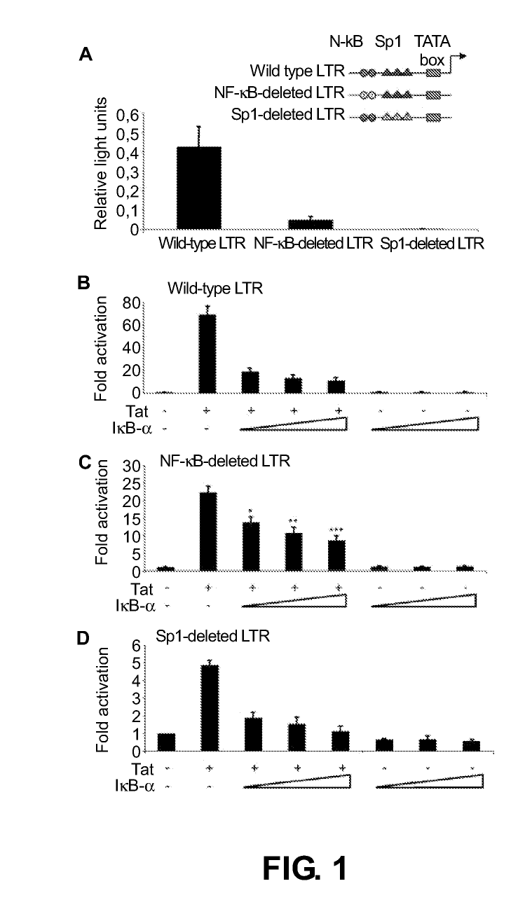

The results illustrated in FIG. 1 show that IκB-α inhibits the transactivation of HIV-1 LTR by Tat independently of the NF-κB repression. In particular, as shown in FIG. 1, the deletion of the NF-κB or Sp1 sites significantly reduced the basal expression (FIG. 1A) and the Tat-mediated transactivation (FIG. 1, B-D) of the HIV-1 LTR. IκB-α inhibited the Tat transcriptional activity in a dose-dependent manner up to 80% for the wild type LTR (FIG. 1B) and 60% in the case of NF-κB-deleted LTR (FIG. 1C).

The evidence that IκB-α inhibited the Tat-mediated transactivation of the LTR in the absence of the NF-κB enhancer underscored the existence of mechanisms of LTR inhibition distinct from NF-κB repression. IκB-α completely repressed the Tat-mediated transactivation of the Sp1-deleted LTR (FIG. 1D). A possible explanation that is not intended to be limiting and is provided for completeness of description and guidance to the skilled person only, this strong inhibition was likely caused by repression of both NF-κB-dependent and independent transactivation of the LTR.

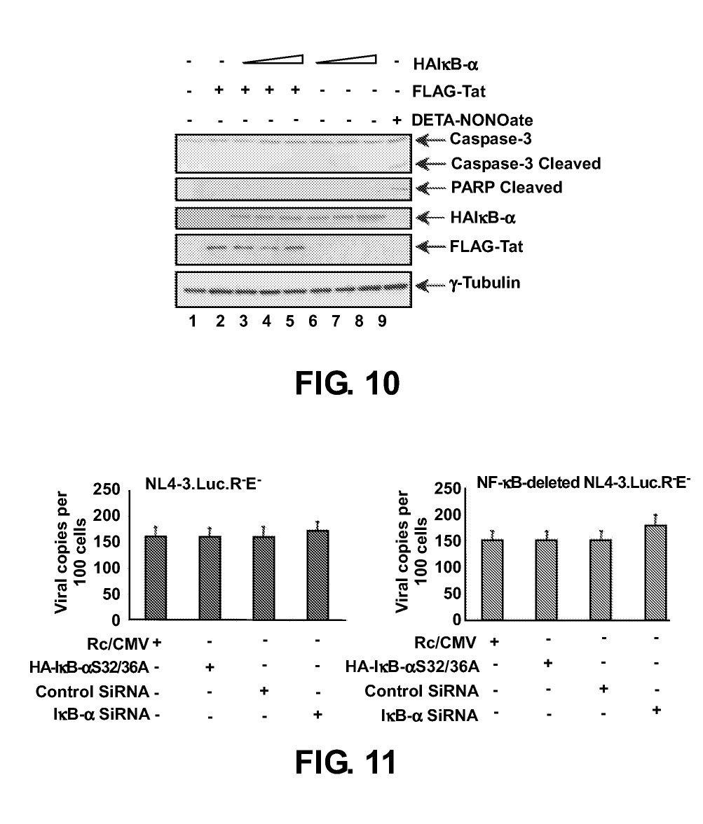

To verify a possible pro-apoptotic activity, HeLa cells (3×10⁵) were transfected with or without p3XFLAG-CMV-Tat (0.5 μg) and pCMV4-HA-IκB-α (0.5, 1 and 2 μg) and 48 h post-transfection the cell extracts (20 μg) were analyzed by Western blotting with anti-Caspase-3, cleaved PARP, anti-HA, anti-FLAG and anti-γ-tubulin antibodies. As control of Caspase-3 and PARP cleavages, extracts (20 μg) from HeLa cells 24 h-stimulated with DETA-NONOate (1 mg/ml) were analyzed. The results illustrated in FIG. 10 shows that IκB-α does not induce apoptosis in transfected HeLa cells. Therefore the LTR inhibition was not a consequence of pro-apoptotic activity of IκB-α because the cleavage of caspase-3 and poly(ADP-ribose)polymerase was undetected in IκB-α-transfected cells.

Next, the effect of IκB-α on Tat was analyzed in the absence of NF-κB activity. To this end, the expression of the NF-κB-deleted LTR was analyzed in MEFs lacking the p50 and p65 subunits of NF-κB. Because the murine cyclin T1 does not allow the generation of the P-TEFb/Tat/transactivation-responsive region complex for efficient transcriptional elongation (53), p50^{-/-} p65^{-/-} MEFs were transfected with or without the hCycT1.

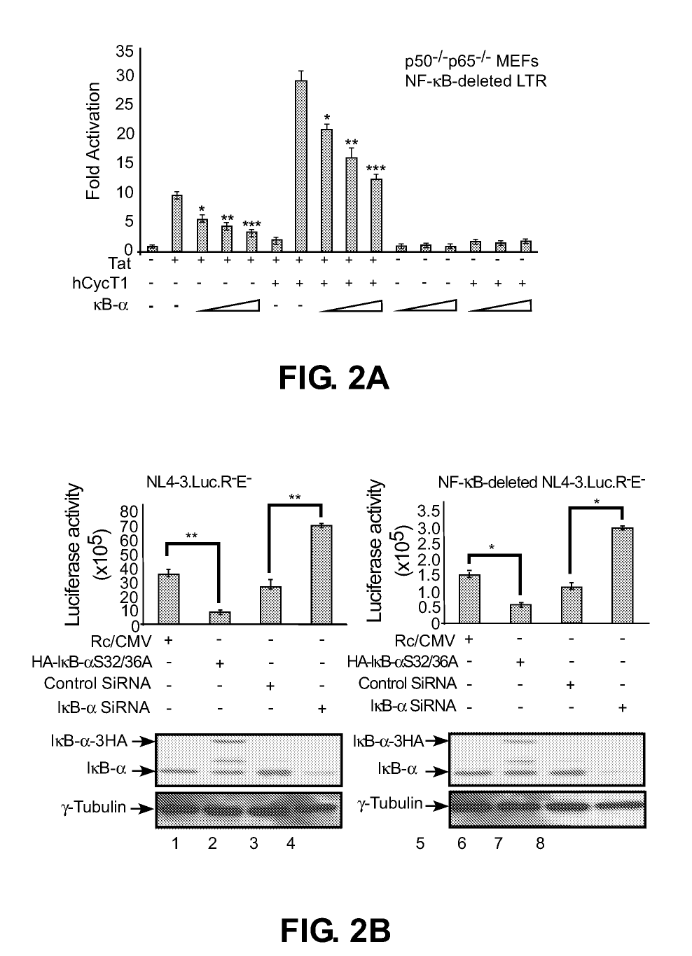

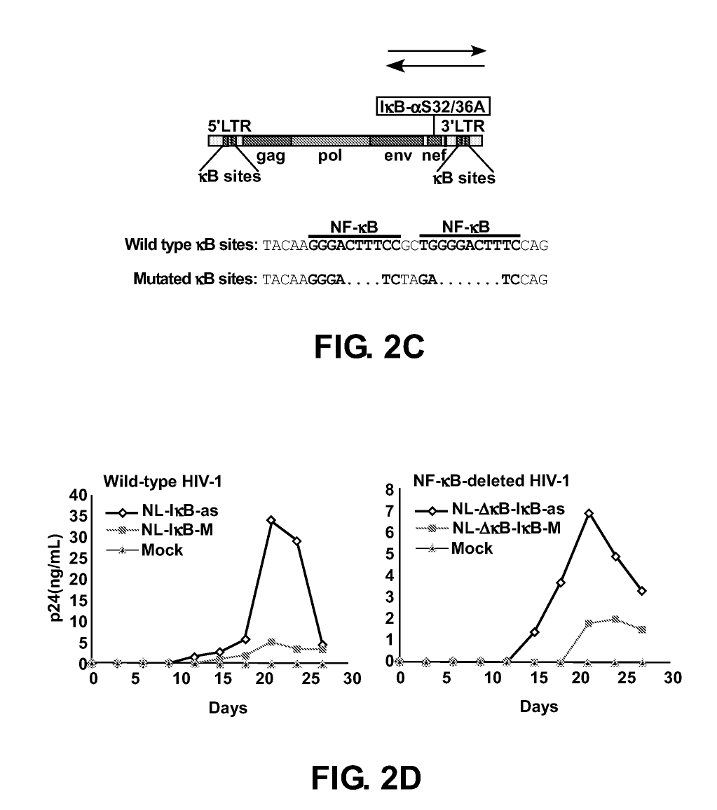

In a first series of experiments, p50^{-/-} p65^{-/-} MEFs (3×10⁵) were transfected with the NF-κB-deleted pLTRluc (0.5 μg), with or without p3XFLAG-CMV-Tat (0.5 μg), pRc/CMV-HA-hCycT1 (0.5 μg), and pCMV4-HA-IκB-α (0.5, 1, and 2 μg). The luciferase activity was measured 48 h post-transfection. Fold activation was calculated relative to transfection in the absence of Tat, hCycT1, and IκB-α expression plasmids. The results illustrated in FIG. 2A show that IκB-α inhibits the expression and replication of NF-κB-deleted viruses. A. In particular, the results shown in FIG. 2A indicate that IκB-α significantly inhibited the Tat-mediated transactivation of the NF-κB-deleted LTR. Additionally those results indicate that Tat inhibition by IκB-α occurs in a dose-dependent manner in presence or absence of hCycT1 (FIG. 2A), which rules out the possibility that IκB-α repressed the Tat activity by interaction with hCycT1

Further, the effect of IκB-α on the expression of the single-cycle replication virus NL4-3.Luc.R-E-carrying the wild type or NF-κB-deleted LTR was analyzed. In particular, in a second series of experiments, Jurkat cells were transfected with the proteolysis-resistant mutant IκB-α S32/36A or with IκB-α siRNA to up-regulate or down-regulate the intracellular levels of IκB-α, respectively. Transfected cells were infected with VSV-G-pseudotyped NL4-3.Luc.R⁻E⁻ virions that carry the wild type or NF-κB-deleted LTR. More particularly, Jurkat cells (4×10⁶) were electroporated with pCMV4-HA-IκB-α or empty vector (30 μg), IκB-α siRNA, or control siRNA (500 pmol) and infected with VSV-G-pseudotyped NL4-3.Luc.R-E-virions that carry the wild type LTR or the NF-κB-deleted LTR (500 ng of p24). Virus production was monitored by measuring the luciferase activity in cell extracts 48 h post-infection. The expression level of IκB-α was detected in cell extracts by Western blotting with anti-IκB-α C-15. The results illustrated in FIG. 2B show that virion production was significantly reduced by hyperexpression of IκB-α and increased by knocking down the endogenous IκB-α with IκB-α siRNA in both infections with the wild type (FIG. 2B, left panel) or the NF-κB-deleted virus (FIG. 2B, right panel).

To analyze the effect of IκB-α on the HIV-1 replication in the absence of the NF-κB-binding sites of the HIV-1 LTR, the viral plasmids pNL-ΔκB-IκB-M and pNL-Δ κB-IκB-as were generated, which carry the IκB-α S32/36A-FLAG cDNA inserted into the nef region in sense or antisense orientation, respectively, and were deleted of the two tandem κB sites in the LTR.

In particular, to achieve this purpose, Jurkat cells (5×10⁴) were infected with equal amounts (0.3 ng of p24) of the wild type LTR viruses, NL-IκB-M and NL-IκB-as, or NF-κB-deleted LTR viruses, NL-ΔκB-IκB-M and NL-ΔκB-IκB-as. The viral production was measured as p24 level in culture supernatants. The resulting recombinant HIV-1 plasmids were the NF-κB-deleted derivatives of pNL-IκB-M and pNL-IκB-as (1), which express or do not express, respectively, IκB-α S32/36A-FLAG. The schematic representation of the viral genome of NL-IκB-M and NL-IκB-as carrying the wild type LTR or NL-ΔκB-IκB-M and NL-ΔκB-IκB-as carrying the NF-κB-deleted LTR, is illustrated in FIG. 2C.

The results of these experiments, illustrated in FIG. 2D, show that, as previously reported (1), NL-IκB-M was potently attenuated as compared with the control NL-IκB-as because of the IκB-α S32/36A expression (FIG. 2D, left panel). Additionally, in the case of NF-κB-deleted viruses, a significant attenuation of NL-ΔκB-IκB-M was also observed as compared with the control NL-ΔκB-IκB-as (FIG. 2D, right panel). These results indicate that IκB-α inhibited the HIV-1 replication independently of the NF-κB enhancer in the HIV-1 LTR and supported the evidence of additional mechanisms of HIV-1 inhibition by IκB-α other than NF-κB repression.

The integration of VSV-G-pseudotyped NL4-3.Luc.R-E-virions was also analyzed. In particular, Jurkat cells (4×10⁶) were electroporated with pCMV4-HA-IκB-α or empty vector (30 μg), IκB-α siRNA or control siRNA (500 pmol), and infected with the wild type or NF-κB-deleted VSV-Gpseudotyped NL4-3.Luc.R-E- (500 ng of p24). The integrated viral copies were measured in the genomic DNA of infected cells by Real-Time PCR. The results illustrated in FIG. 11 show no difference in the number of integrated virus among the different samples (FIG. 11). These results suggest that the levels of endogenous IκB-α inversely affected the expression of the integrated HIV-1 genome independently of the presence of the NF-κB-binding sites in the HIV-1 LTR.

### Example 2

### Tat Inhibition by IκB-α Derivatives

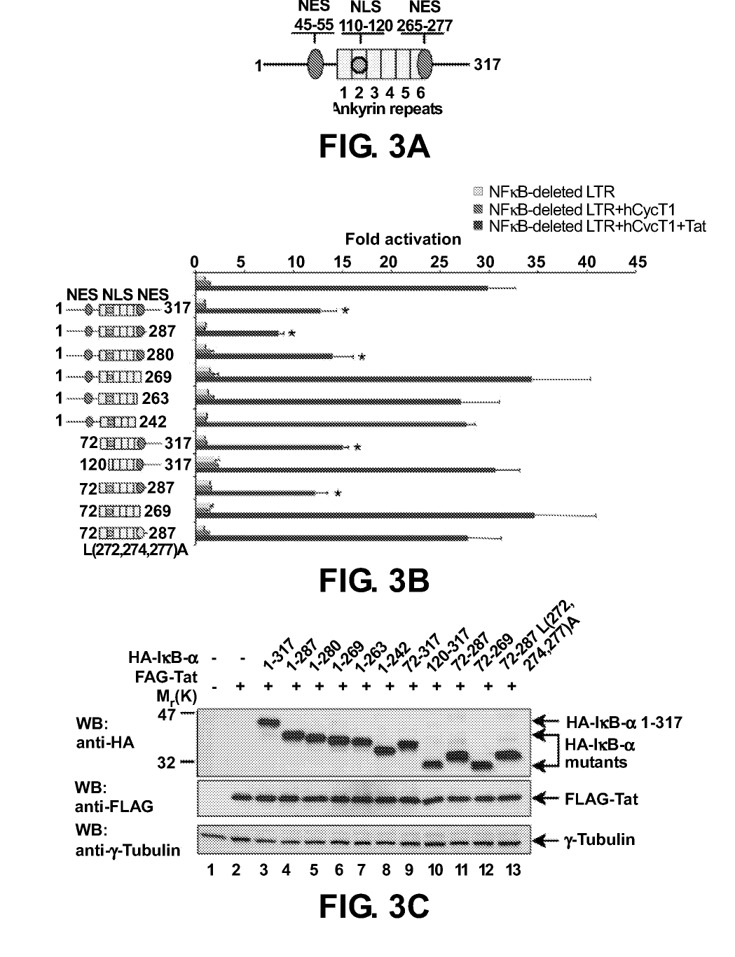

The sequence of IκB-α encompassing amino acids 1-317 contains six ankyrins (amino acids 72-287), the NLS (amino acids 110-120), the N-NES (amino acids 45-55), and the C-NES (amino acids 265-277) (FIG. 3A). To map the IκB-α domains required for Tat inhibition independently of NF-κB repression, the activity of IκB-α mutants was analyzed in p50^{-/-} p65^{-/-} MEFs by transient expression of the NF-κB-deleted LTR and Tat. In particular, p50^{-/-} p65^{-/-} MEFs (3×10⁵) were transfected with the NF-κB-deleted LTRluc (0.5 μg) in presence or absence of p3XFLAG-CMV-Tat (0.5 μg), pRc/CMV-HA-hCycT1 (0.5 μg), and pCMV4-HA-IκB-α 1-317 or the indicated IκB-α mutants (2 μg). The luciferase activity was measured in cell extracts 48 h post-transfection. Fold activation was calculated relative to transfection in the absence of Tat, hCycT1, and IκB-α expression plasmids.

The results illustrated in FIG. 3B shows that the sequence of IκB-α extending from amino acids 72 to 287 inhibits Tat. In particular, as shown in FIG. 3B, the IκB-α mutants that were progressively deleted of the carboxyl-terminal from amino acids 317 to 280 significantly inhibited the Tat activity, whereas no inhibition was induced by IκB-α 1-269 deleted of the C-NES (FIG. 3B). Further, deletions of the carboxyl-terminal of IκB-α from amino acids 269 to 242 did not affect the Tat activity (FIG. 3B). IκB-α 72-317 lacking the amino-terminal sequence from amino acids 1 to 72 significantly inhibited Tat, whereas IκB-α 120-317, which was deleted of the NLS, lost the inhibitory activity (FIG. 3B). These results indicated that the sequences of IκB-α from amino acids 72 to 120 (overlapping the NLS) and from amino acids 269 to 280 (overlapping the C-NES) were both required for Tat inhibition.

This was confirmed by experiments where the mutant IκB-α 72-287, which contains both the NLS and C-NES, inhibited Tat, whereas the mutants IκB-α 72-269 and IκB-α 72-287 L(272,274,277)A, which carry deletion or base pair substitutions of critical leucine residues of the C-NES sequence [8], respectively, failed to inhibit Tat (FIG. 3B).

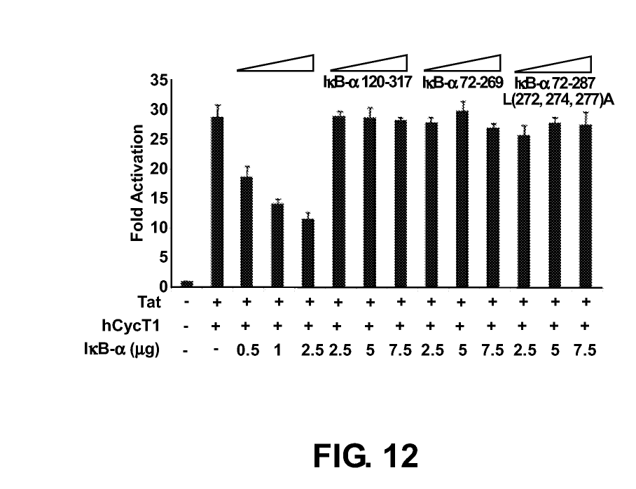

Lack of inhibition was confirmed at higher doses of IκB-α 120-317, IκB-α 72-269, and IκB-α 72-287 L(272,274,277)A. In particular, p50^{-/-} p65^{-/-} MEFs (3×10⁵) were transfected with the NF-κB-deleted LTRluc (0.5 μg) in presence or absence of p3XFLAG-CMV-Tat (0.5 μg), pRc/CMVHA-hCycT1 (0.5 μg) and pCMV4-HA-IκB-α 1-317 or the mutants IκB-α 120-317, IκB-α 72-269, IκB-α 72-287L(272, 274, 277)A at the indicated doses. The luciferase activity was measured in cell extracts 48 h post-transfection. Fold activation was calculated relative to transfection in the absence of Tat, hCycT1 and IκB-α expression plasmids. The results illustrated in FIG. 12 show that the IκB-α mutants lacking the NLS or the C-NES do not inhibit the Tat transactivation of the NF-κB-deleted HIV-1 LTR.

The IκB-α mutants were all expressed in cell extracts, and no correlation was found between the level of expression and the inhibitory activity. In particular, cell extracts (20 μg) of p50^{-/-} p65^{-/-} MEFs transfected with p3XFLAG-CMV-Tat and pCMV4-HA-IκB-α mutants as shown in B were analyzed by Western blotting (WB) for the expression of transfected genes. These results, illustrated in FIG. 3C, demonstrated that the minimal sequence of IκB-α required for Tat inhibition spanned from amino acids 72 to 287. This region encompasses the six ankyrins of IκB-α including the NLS and C-NES.

### Example 3

### Tat /IκB-α Interaction: Binding of IκB-α to the Arginine-Rich Domain of Tat

To test whether IκB-α physically interacts with Tat, the GST pulldown assay was performed with extracts from cells transfected with pCMV4-HA-IκB-α.

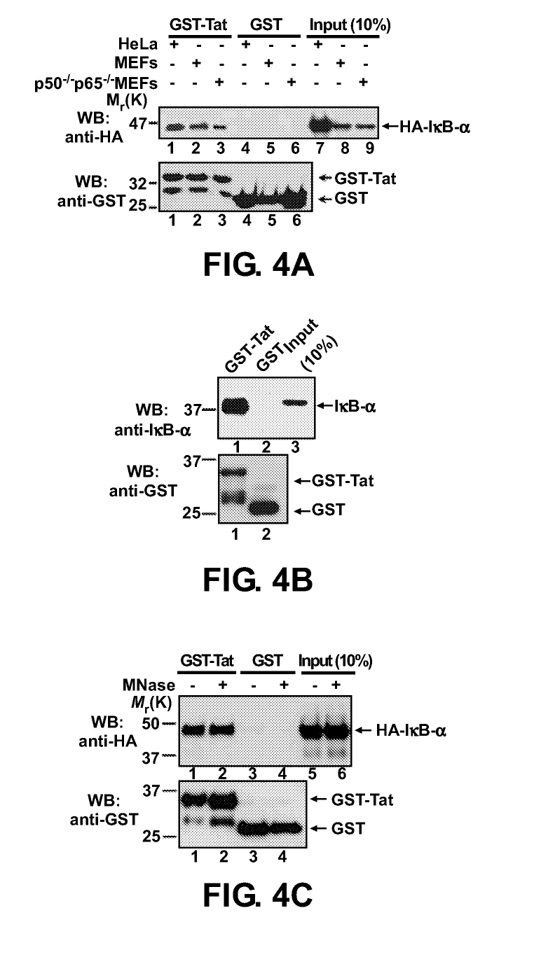

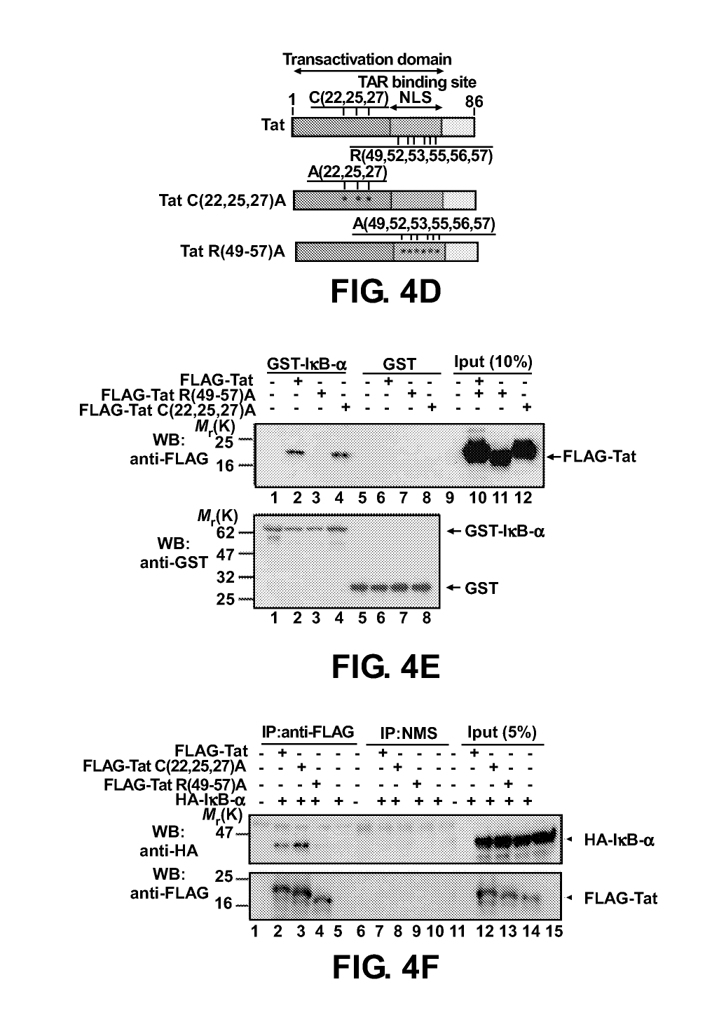

In particular, in a first series of experiments HeLa cells, MEFs and p50^{-/-} p65^{-/-} MEFs (1×10⁶) were transfected with pCMV4-HA-IκB-α (5 μg), and cell extracts were incubated with GST-Tat or GST. Following GST pulldown, the protein complexes were analyzed by Western blotting (WB) with anti-HA and anti-GST antibodies. The results illustrated in FIG. 4A, show that GST-Tat retained IκB-α expressed in HeLa and MEFs (FIG. 4A, lanes 1 and 2). The binding of Tat with IκB-α was also observed in p50^{-/-} p65^{-/-} MEFs (FIG. 4A, lane 3), which ruled out that IκB-α and Tat were recruited in the same complex by associating with the p50 and p65 subunits of NF-κB. IκB-α was not retained by GST protein (FIG. 4A, lanes 4-6).

In a second series of experiments, cell extracts (1 mg) from HeLa cells were incubated with GST-Tat or GST (50 μg). Following GST pulldown, the protein complexes were analyzed by Western blotting with anti-IκB-α (C-15) and anti-GST antibodies. The results illustrated in FIG. 4B, show that the association of endogenous IκB-α with GST-Tat is also observed in HeLa extracts (FIG. 4B, lane 1).

In a third series of experiments, HeLa cells (1×10⁶) were transfected with pCMV4-HA-IκB-α (5 μg), and cell extracts (200 μg) were treated with micrococcal nuclease for 30 min at 28° C. or left untreated. The extracts were incubated with GST-Tat or GST. After GST pulldown the protein complexes were analyzed by Western blotting with anti-HA and anti-GST antibodies. The results illustrated in FIG. 4C, show that treatment of the cellular extracts with micrococcal nuclease did not affect the binding of IκB-α with Tat (FIG. 4C, lane 2), thus ruling out the possibility that the association of the two proteins was bridged by nucleic acids.

To map the Tat domain that binds to IκB-α, GST-IκB-α was incubated with extracts from HeLa cells transfected with the wild type Tat or the mutants Tat C(22,25,27)A and Tat R(49-57)A fused to the FLAG epitope. In particular, in a fourth series of experiments, HeLa cells (1×10⁶) were transfected with p3XFLAG-CMV-Tat, p3XFLAG-CMV-Tat R(49-57)A, or p3XFLAG-CMV-Tat C(22,25,27)A (5 μg). Forty-eight hours post-transfection the cell extracts were incubated with GST-IκB-α or GST conjugated with glutathione-Sepharose. The protein complexes were recovered by GST pulldown, separated by 10% SDS-PAGE, and analyzed by Western blotting with anti-FLAG and anti-GST antibodies. A schematic representation of wild type Tat and the mutants Tat C(22,25,27)A and Tat R(49-57)A is shown in FIG. 4D.

The results illustrated in FIG. 4E show that in pulldown assay, GST-IκB-α retained the wild type Tat and Tat C(22,25,27)A (FIG. 4E, lanes 2 and 4), whereas it did not bind to Tat R(49-57)A (FIG. 4E, lane 3). Tat was not retained by GST alone (FIG. 4E, lanes 6-8).

The association of IκB-α with Tat was further tested by in vivo immunoprecipitation with extracts from HeLa cells transfected with the plasmids expressing FLAG-Tat and HA-IκB-α. In particular, in a fifth series of experiments, HeLa cells (1×10⁶) were transfected with p3XFLAG-CMV-Tat, p3XFLAG-CMV-Tat R(49-57)A, or p3XFLAG-CMV-Tat C(22,25,27)A (5 μg) in presence or absence of pCMV4-HA-IκB-α (5 μg). The cell extracts were performed 48 h post-transfection and immunoprecipitated (IP) with anti-FLAG or normal mouse serum. The immunocomplexes were separated by 10% SDS-PAGE and analyzed by Western blotting with anti-HA and anti-FLAG antibodies.

The results illustrated in FIG. 4F show that IκB-α immunoprecipitated with the wild type Tat and Tat C(22,25,27)A (FIG. 4F, lanes 2 and 3), whereas it did not associate with Tat R(49-57)A (FIG. 4F, lane 4). Altogether, these results indicate that the arginine-rich region of Tat encompassing amino acids 49-57 is required for the association with IκB-α.

Overall, the above results, illustrated in FIG. 4, show that IκB-α binds to the arginine-rich domain of Tat.

### Example 4

### Tat /IκB-α Interaction: Binding of Tat to the Sixth Ankyrin of IκB-α

To determine the sequence of IκB-α binding to Tat, [35S]methionine-labeled IκB-α mutants were incubated with GST-Tat or GST.

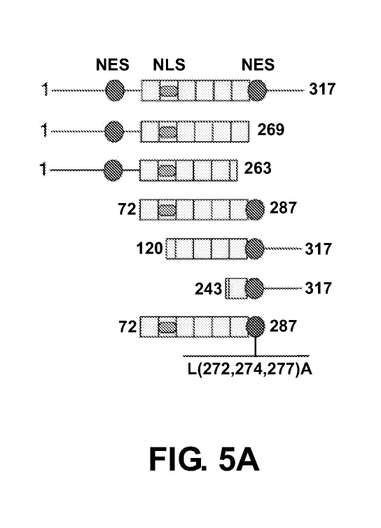

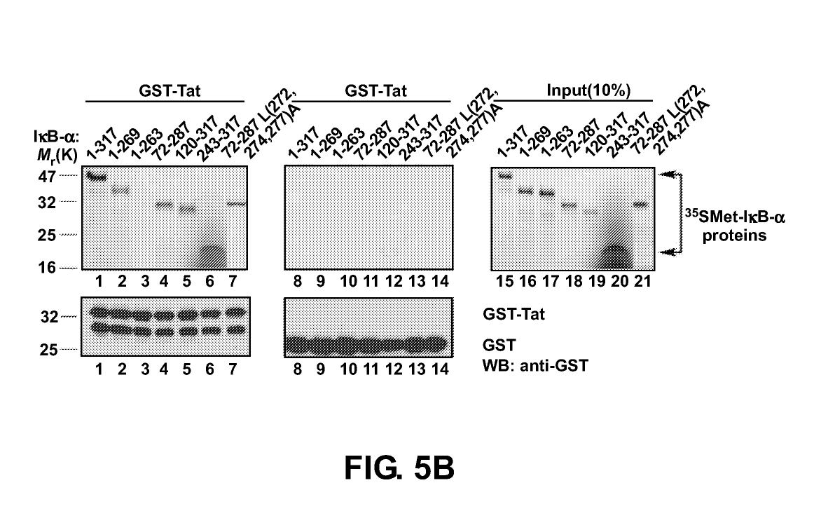

In particular, [³⁵S]methionine-labeled IκB-α proteins were incubated with GST-Tat or GST. Following GST pulldown, the protein complexes were separated by 12% SDS-PAGE and analyzed by autoradiography and by Western blotting with anti-GST antibody. A schematic representation of IκB-α proteins used for the GST-Tat pulldown is illustrated in FIG. 5A. The results illustrated in FIG. 5B show that Tat retained IκB-α 1-317 and IκB-α 1-269 (FIG. 5B, lanes 1 and 2), whereas it did not bind to IκB-α 1-263 (FIG. 5B, lane 3). The mutants IκB-α 72-287, IκB-α 120-317, IκB-α 243-317, and IκB-α 72-287 L(272, 274, 277)A were efficient binders of Tat (FIG. 5B, lanes 4-7). As control, GST tested negative for the binding to labeled proteins (FIG. 5B, lanes 8-14). These results indicated that Tat binds to the sixth ankyrin of IκB-α and that the IκB-α sequence from amino acids 263 to 269 within the sixth ankyrin of IκB-α was required for binding to Tat.

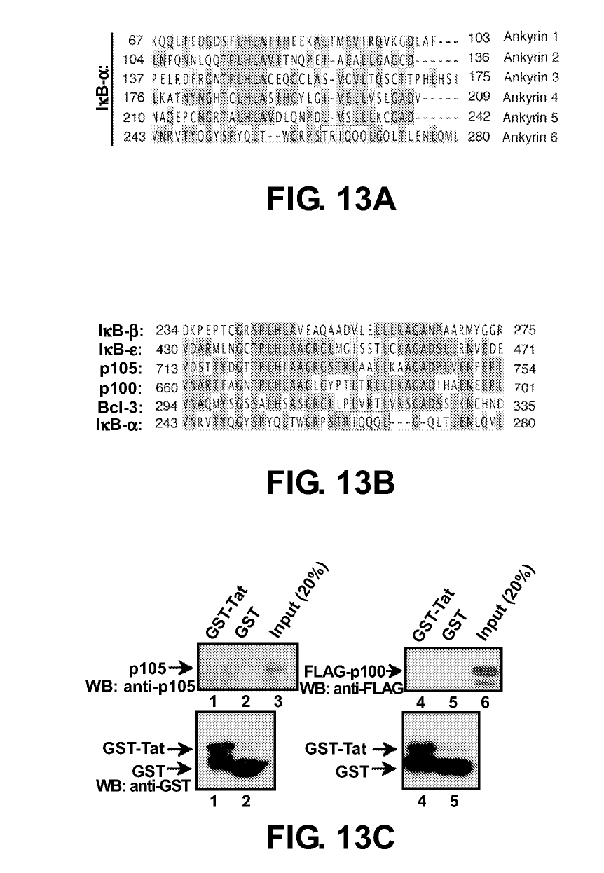

A comparison performed by CLUSTALW-based multiple sequence alignment (align.genome.jp), shows that the sixth ankyrin of IκB-α, which appear to bind to Tat, includes a unique diverged sequence as compared to other ankyrins. More particularly, such a comparison show that the amino acid sequence of the IκB-α sixth ankyrin, is very divergent from the other five ankyrins of IκB-α and the ankyrins of the human IκB family (p100, p105, IκB-γ, IκB-ε, and Bcl-3). Reference is made to the schematics illustrated in FIGS. 13A and B). More particularly, the sequence TRIQQQL (SEQ ID NO: 14) (amino acids 263-269 of IκB-α), which is present in the sixth ankyrin and is required for the binding to Tat (FIG. 5B), is absent in ankyrins 1-5 of IκB-α as well as in the ankyrins of the IκB family members (FIGS. 13, A and B).

A more extended analysis by using FUZZPRO (bioweb.pasteur.fr/seqanal/interfaces/fuzzpro.html) failed to identify the TRIQQQL motif in the ankyrins of the human proteome except in the sixth ankyrin of IκB-α. The ability of the sequence TRIQQQL (SEQ ID NO: 14) to bind tat was then tested by a series of experiments. In particular, in vitro translated p105 and FLAG-p100 (two members of the IκB family showing the highest identity with the sixth ankyrin of IκB-α) were incubated with GST-Tat or GST. Following GST pull-down, the protein complexes were separated by 12% SDS-PAGE and analyzed by Western blotting with anti-p105, anti-FLAG and anti-GST antibody. The results illustrated in FIG. 13C, show that p100 and p105 were unable to bind to Tat (FIG. 13C, lanes 1 and 4).

These results suggest that the sixth ankyrin of IκB-α, contains a unique diverged sequence as compared with other ankyrins, which might represent a privileged target site for Tat binding. Alternatively, this sequence might contribute to stabilize a peculiar structural domain required for the binding to Tat.

### Example 5

### IκB-α Exports Tat from the Nucleus to the Cytoplasm

The cellular distribution of IκB-α and Tat was visualized by confocal fluorescence microscopy. HeLa cells were transfected with plasmids expressing FLAG-Tat and HA-IκB-α.

In particular, in a first series of experiments HeLa cells (5×10⁵) were transfected with p3XFLAG-CMV-Tat (3 μg) in the presence or absence of pCMV4-HA-IκB-α 1-317, pCMV4-HA-IκB-α 120-317, pCMV4-HA-IκB-α 1-269, pCMV4-HA-IκB-α 72-287, pCMV4-HA-IκB-α 72-269, or pCMV4-HA-IκB-α 72-287 L(272,274,277)A (3 μg). The cells were analyzed by confocal microscopy as described under “Experimental Procedures.”

In a second series of experiments, total extracts (25 μg) from transfected HeLa cells from the first series of experiments were analyzed by Western blotting (WB) with anti-HA, anti-FLAG, and anti-γ-tubulin antibodies.

The results of these first and second series of experiments were confirmed by fluorescence-based analysis of Tat and IκB-α. In particular, a fluorescence-based evaluation of FLAG-Tat and HA-IκB-α was performed in HeLa cells upon transfection as detailed under “Experimental Procedures.” Thirty cells were recorded and analyzed for each transfection. The relative nuclear or cytoplasmic fluorescence was evaluated as the ratio between the mean nuclear or cytoplasmic fluorescence and the mean fluorescence of the whole cell.

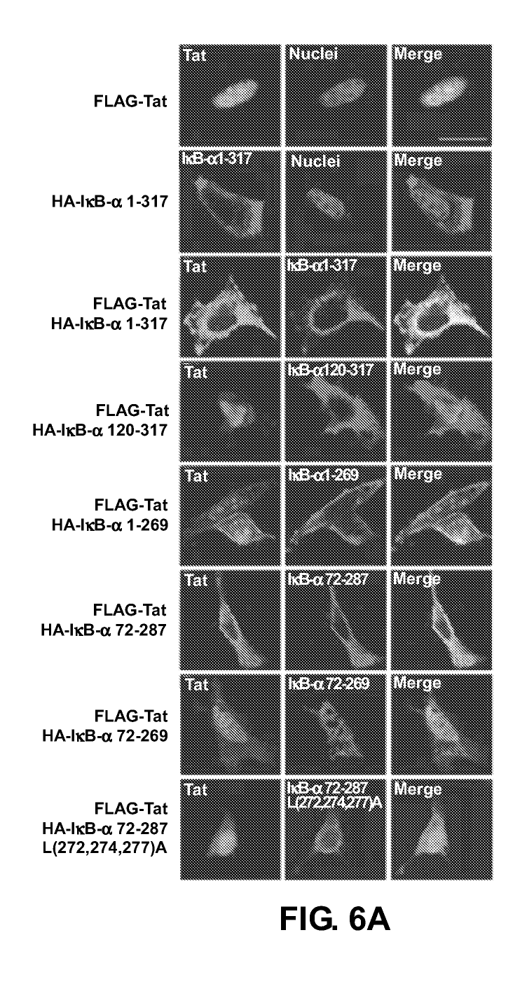

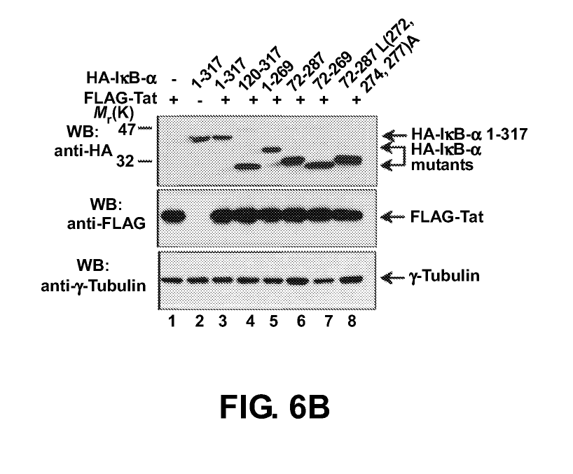

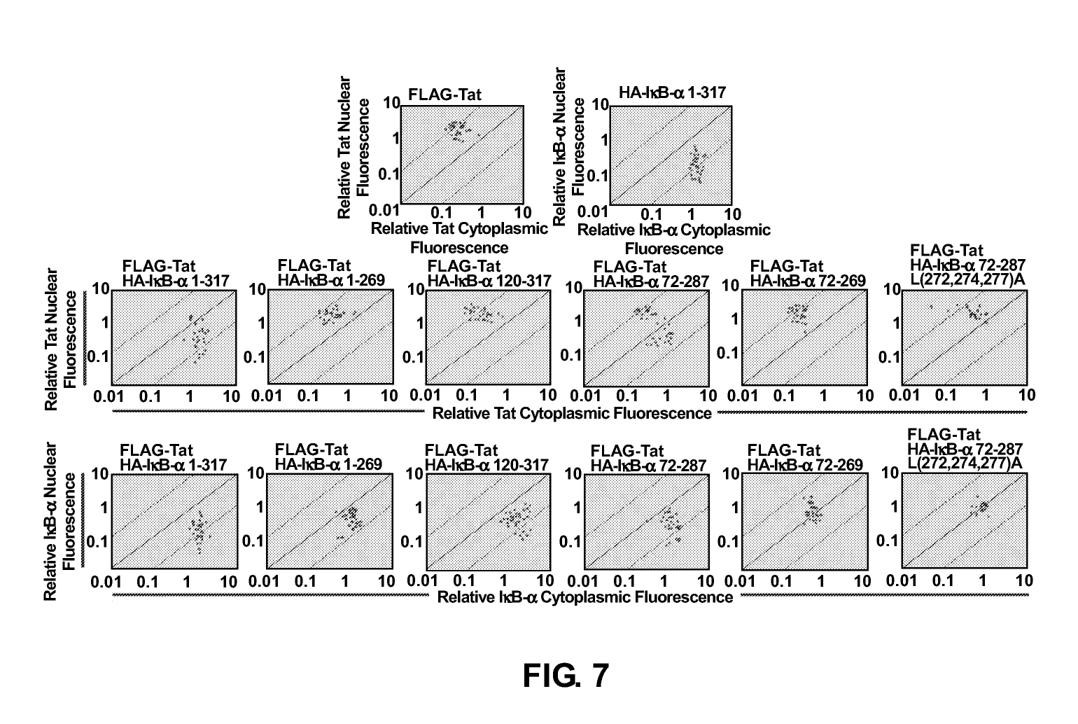

The results of the above experiments illustrated in FIGS. 6 and 7 provided several indication on the cell localization of Tat and IκB-α. In particular, when singularly transfected, Tat was nuclear, whereas IκB-α 1-317 was mostly cytoplasmic (FIG. 6A). This was confirmed by the fluorescence-based analysis of 30 cells for each transfection (FIG. 7).

When co-transfected, Tat and IκB-α 1-317 co-localized within the cytoplasmic and perinuclear regions (FIGS. 6A and 7). IκB-α 120-317, lacking both the N-NES and the NLS, and IκB-α 1-269, lacking the C-NES, were prevalently cytoplasmic and did not affect the nuclear location of Tat (FIGS. 6A and 7). IκB-α 72-287, lacking the N-NES, was mostly cytoplasmic and promoted the translocation of Tat from the nucleus to the cytoplasm in 50% of the analyzed cells (FIGS. 6A and 7).

IκB-α 72-269 and IκB-α 72-287 L(272,274,277)A, which lacked the N-NES and C-NES, were distributed both in the nucleus and cytoplasm and did not affect the nuclear location of Tat (FIGS. 6A and 7). No significant differences in the intracellular expression levels of the IκB-α mutants were observed in transfected cells (FIG. 6B). FIG. 6 shows that IκB-α promotes the nuclear export of Tat. These results suggested that IκB-α promoted the displacement of Tat from nucleus to cytoplasm and that this activity required the integrity of the NLS and C-NES of IκB-α.

To analyze the role of the nuclear export activity of IκB-α in Tat inhibition, the mutant IκB-α 1-317 N/C NES was generated, which carries crucial base pair substitutions of both the N-NES (I52A,L54A) and C-NES (L272,274,277A), which inactivate the nuclear export activity. In particular in a first series of experiments, HeLa cells (5×10⁵) were transfected with p3XFLAG-CMV-Tat (3 μg) and pCMV4-HA-IκB-α 1-317 N/C NES (3 μg). The cells were analyzed by confocal microscopy as described under “Experimental Procedures.”

In a second series of experiments, the fluorescence-based analysis of FLAG-Tat and HA-IκB-α was performed as detailed above with reference to the results illustrated in FIG. 7C. In particular, p50^{-/-} p65^{-/-} MEFs (3×10⁵) were transfected with the NF-κB-deleted LTRluc (0.5 μg) in presence or absence of p3XFLAG-CMV-Tat (0.5 μg), pRc/CMV-HA-hCycT1 (0.5 μg), and pCMV4-HA-IκB-α 1-317 or pCMV4-HA-IκB-α N/C NES (2 μg). The luciferase activity was measured in cell extracts 48 h post-transfection. Fold activation was calculated relative to transfection in the absence of Tat, hCycT1, and IκB-α expression plasmids.

In a third series of experiments [³⁵S]methionine-labeled IκB-α wild type and IκB-α N/C NES were incubated with GST-Tat or GST. Following GST pulldown, the protein complexes were separated by 12% SDS-PAGE and analyzed by autoradiography and by Western blotting with anti-GST antibody.

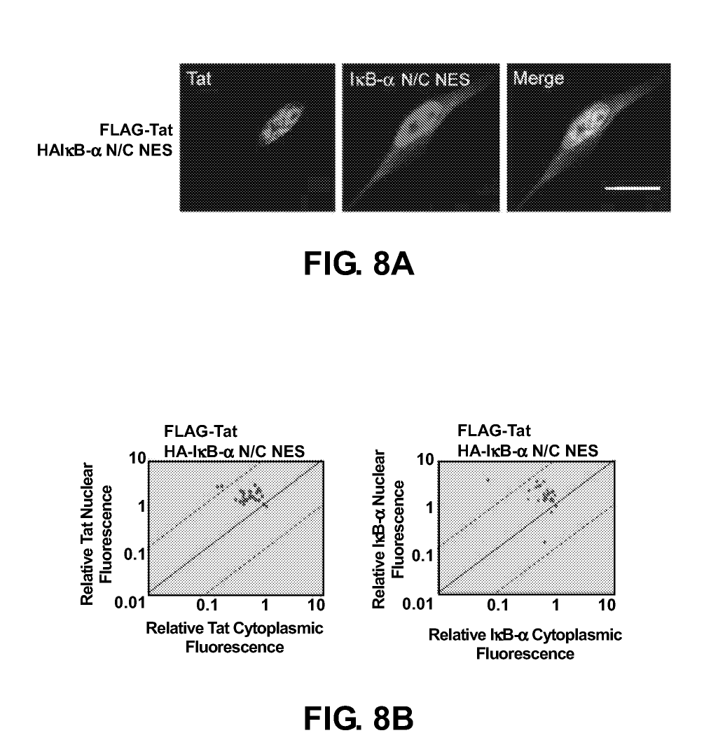

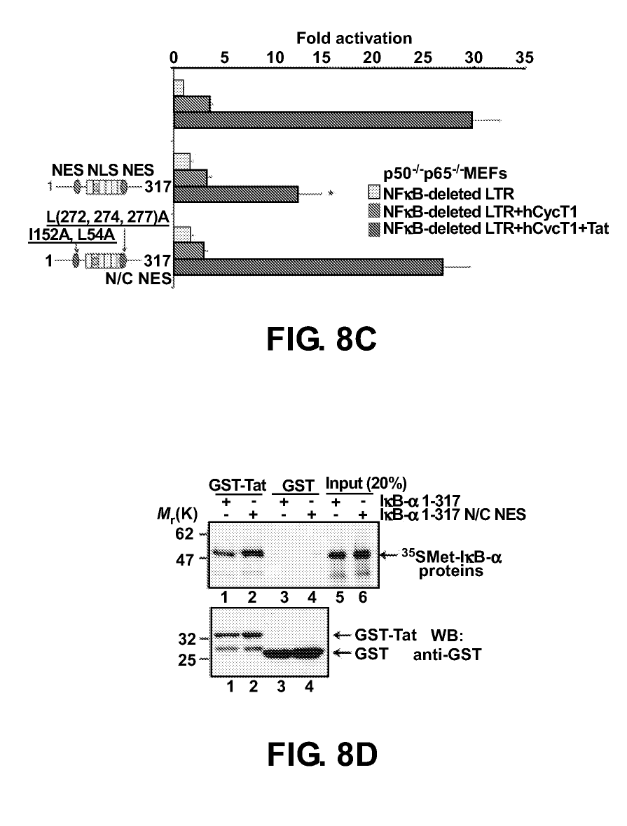

The results illustrated in FIG. 8 show that the nuclear export activity of IκB-α is required for nuclear export and inhibition of Tat. In particular, the results indicated that IκB-α N/C NES was prevalently distributed in the nucleus and did not affect the nuclear location of Tat (FIGS. 8, A and B). Moreover, IκB-α N/C NES did not repress the Tat-mediated transactivation of the NF-κB-deleted LTR (FIG. 8C), although it was able to bind to Tat in GST-pull down (FIG. 8D, lane 2). These results confirmed that IκB-α inhibited Tat through the nuclear export to the cytoplasm.

The effect of leptomycin B, a nuclear export inhibitor, on the inhibition of Tat by IκB-α was also verified. In particular, p50^{-/-} p65^{-/-} MEFs (3×10⁵) were transfected with the NF-κB-deleted pLTRluc (0.5 μg) in presence or absence of p3XFLAG-CMV-Tat (0.5 μg), pRc/CMV-HA-hCycT1 (0.5 μg) and pCMV4-HA-IκB-α 1-317. Cells were cultured with LMB (20 nM) immediately after transfection, or left untreated. The luciferase activity was measured in cell extracts 18 h post-transfection. Fold activation was calculated relative to transfection in the absence of Tat, hCycT1 and IκB-α expression plasmids.

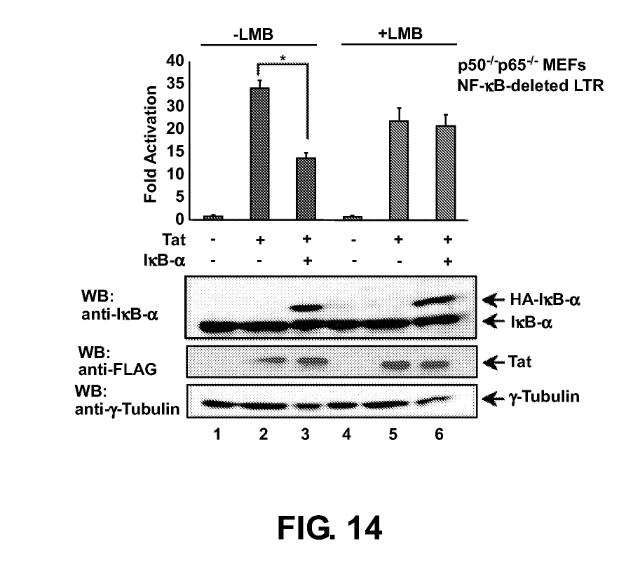

The results illustrated in FIG. 14 show that leptomycin B, a nuclear export inhibitor, causes the loss of Tat inhibition by IκB-α. In particular, the results indicate that in p50^{-/-} p65^{-/-} MEFs, leptomycin B did not affect significantly the level of Tat-mediated transactivation of the NF-κB-deleted LTR (FIG. 14, lanes 2 and 5), whereas it caused the loss of Tat inhibition by the transfected IκB-α (FIG. 14, comparison between lanes 2-3 and lanes 5-6). These results indicate that the leptomycin B-mediated arrest of nuclear export released Tat from the IκB-α inhibition.

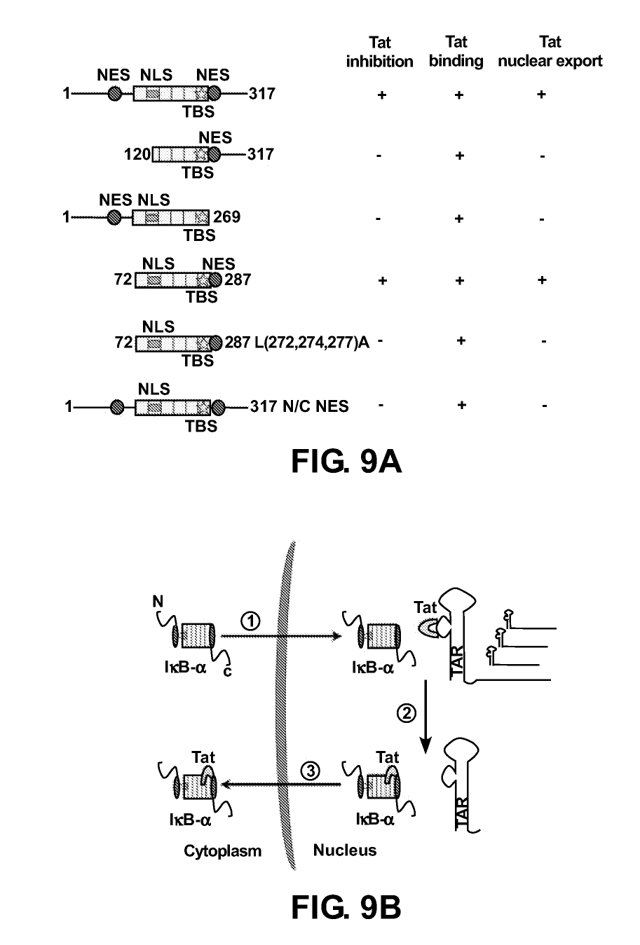

The studies and experiments exemplified in the present section out provide further insight into the mechanisms of HIV-1 inhibition by the IκB-α repressor, particularly by the IκB-α-derived polypeptide inhibitor herein disclosed. A possible mechanism of Tat inhibition by IκB-α, herein indicated for completeness of description and guidance only and is not intended to be limiting the scope of the present disclosure is illustrated in the schematic model FIG. 9.

In particular, as illustrated in FIG. 9, it appears that at least in some embodiments, IκB-α represses Tat activity independently of the NF-κB inhibitory activity by physical association and displacement of Tat from the nucleus to the cytoplasm. Also in some embodiments, the association of IκB-α with the arginine rich domain of Tat is not sufficient to interfere with the nuclear distribution and the transcriptional activity of Tat. In particular, the mutants IκB-α 120-317 and IκB-α 1-269 appear to bind to Tat without affecting the nuclear location and transcriptional activity of the viral transactivator (FIG. 9A). Instead, the inhibition of Tat correlates with the nuclear export activity of IκB-α, which requires both the NLS (amino acids 110-120) and the C-NES (amino acids 265-277) together with the binding site for Tat (amino acids 263-269) (FIG. 9A). Consistent with this evidence, the mutant IκB-α N/C NES, which contains the full-length sequence of IκB-α but lacks the nuclear export signals, does not affect the nuclear location and the transcriptional activity of Tat (FIG. 9A). Altogether, these results suggest that IκB-α binds to Tat in the nucleus and exports the viral transactivator to the cytoplasm, where the complex IκB-α/Tat is mostly retained (FIG. 9B). In particular, it appears that a possible mechanism for IκB-α repression may involve three steps: the IκB-α repressor enters in the nucleus (step 1), where it associates to Tat (step 2) and exports the viral transactivator to the cytoplasm (step 3). The nuclear localization signal, the carboxyl-terminal nuclear export signal, and the Tat-binding site of IκB-α are required for the nuclear export of Tat.

The evidence that IκB-α inhibits the transcriptional activity of Tat raises the question of why the endogenous IκB-α does not counteract the viral expression in HIV-1-infected cells. Indeed, IκB-α is subjected to persistent proteolysis in the course of HIV-1 infection. The HIV-1 entry through the gp120 envelope protein binding to CD4 receptor activates the IκB kinase complex, which promotes the proteolysis of IκB-α. This event leads to the transcriptional activation of NF-κB-dependent genes, including the HIV-1 genome and pro-inflammatory genes, which in turn sustain the proteolysis of IκB-α and the activation of NF-κB. In particular, Tat activates NF-κB by inducing the degradation of IκB-α, the up-regulation of NIK, and the transactivation of inflammatory cytokines.

The physical and functional interaction of IκB-α with Tat discloses a novel mechanism of HIV-1 transcriptional regulation. In particular, the inhibitory sequence of IκB-α (amino acids 72-287) identified by the present inventors represents a novel peptide-based inhibitor acting at the transcriptional step of the HIV-1 life cycle.

The examples set forth above are provided to give those of ordinary skill in the art a complete disclosure and description of how to make and use the embodiments of the polypeptides, compositions and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

The entire disclosure of each document cited (including patents, patent applications, journal articles, abstracts, laboratory manuals, books, or other disclosures) in the Background, Summary, Detailed Description, and Examples is hereby incorporated herein by reference.

The hard copy of the sequence listing submitted herewith and the corresponding computer readable form are both incorporated herein by reference in their entireties.

It is to be understood that the disclosures are not limited to particular compositions or biological systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

The following abbreviations are used throughout the present disclosure: HIV-1, human immunodeficiency virus, type 1; LTR, long terminal repeat; NF-κB, nuclear factor κB; IκB, inhibitor κB; NLS, nuclear localization signal; NES, nuclear export signal; N-NES, amino-terminal NES; C-NES, carboxyl-terminal NES; MEFs, mouse embryonic fibroblasts; PBS, phosphate-buffered saline; DTT, dithiothreitol; GST, glutathione S-transferase; hCycT1, human cyclin Ti; siRNA, small interfering RNA; HA, hemagglutinin.

Although any methods and materials similar or equivalent to those described herein can be used in the practice for testing of the specific examples of appropriate materials and methods are described herein.

A number of embodiments of the disclosure have been described. Nevertheless, it will be understood that various modifications may be made without departing from the spirit and scope of the present disclosure. Accordingly, other embodiments are within the scope of the following claims.

### REFERENCES

1. Quinto, I., Mallardo, M., Baldassarre, F., Scala, G., Englund, G., and Jeang, K. T. (1999) J. Biol. Chem. 274, 17567-17572.
2. Ambrosino, C., Ruocco, M. R., Chen, X., Mallardo, M., Baudi, F., Trematerra, S., Quinto, I., Venuta, S., and Scala, G. (1997) J. Biol. Chem. 272, 14883-14892.
3. Quinto, I., Puca, A., Greenhouse, J., Silvera, P., Yalley-Ogunro, J., Lewis, M. G., Palmieri, C., Trimboli, F., Byrum, R., Adelsberger, J., Venzon, D., Chen, X., and Scala, G. (2004) J. Biol. Chem. 279, 1720-1728.
4. O'Doherty, U., Swiggard, W. J., and Malim, M. H. (2000) J. Virol. 74, 10074-10080.
5. Brussel, A., and Sonigo, P. (2003) J. Virol. 77, 10119-10124.
6. Demarchi, F., d'Adda di Fagagna, F., Falaschi, A., and Giacca, M. (1996) J. Virol. 70, 4427-4437.
7. Liu, W., Quinto, I., Chen, X., Palmieri, C., Rabin, R. L., Schwartz, O. M., Nelson, D. L., and Scala, G. (2001) Nat. Immunol. 2, 939-946.
8. Arenzana-Seisdedos, F., Turpin, P., Rodriguez, M., Thomas, D., Hay, R. T., Virelizier, J. L., and Dargemont, C. (1997) J. Cell Sci. 110, 369-378.
9. Pearson, and Lipman, Proc. Natl. Acad. Sci. USA 85:2444-2448, 1988;
10. Pearson, Methods in Enzymol. 183:63-98, 1990
11. Methods in Enzymology, vol. 266: Computer Methods for Macromolecular Sequence Analysis (1996), ed. Doolittle, Academic Press, Inc., a division of Harcourt Brace & Co., San Diego, Calif., USA.
12. Meth. Mol. Biol. 70: 173-187 (1997).
13. Mol. Biol. 48: 443-453 (1970).
14. Dayhoff et al. (1978) “A model of evolutionary change in proteins” in “Atlas of Protein Sequence and Structure,” Vol. 5, Suppl. 3 (ed. M. O. Dayhoff), pp. 345-352. Natl. Biomed. Res. Found., Washington, D.C.
15. Henikoff et al. (1992) Proc. Nat'l. Acad. Sci. USA 89: 10915-1091

What is claimed is:

1. A polypeptide inhibitor of HIV-1 transcription and replication, the polypeptide comprising
a nuclear localization signal of IκB-α, or a derivative thereof;
a C-terminal nuclear export signal of IκB-α, or a derivative thereof, and
a binding site of IκB-α for an HIV-1 Tat transactivator, or a derivative thereof.

2. The polypeptide inhibitor according to claim 1, the polypeptide comprising an amino acidic sequence consisting of positions 110 to 120 of IκB-α amino acid sequence, or a derivative thereof.

3. The polypeptide inhibitor according to claim 1, the polypeptide comprising an amino acidic sequence consisting of positions 265 to 277 of IκB-α amino acid sequence, or a derivative thereof.

4. The polypeptide inhibitor according to claim 1, the polypeptide comprising an amino acidic sequence consisting of positions 263 to 269 of IκB-α amino acid sequence, or a derivative thereof.

5. The polypeptide inhibitor according to claim 1, the polypeptide comprising an amino acidic sequence consisting of positions 72 to 287 of IκB-α amino acid sequence, or a derivative thereof.

6. The polypeptide inhibitor according to claim 5, the polypeptide comprising an amino acid sequence consisting of SEQ ID NO:1, or a derivative thereof.

7. The polypeptide inhibitor according to claim 6, having an amino acid sequence having an at least 90% identity to SEQ ID NO:1.

8. A method of inhibiting HIV-1 transcription and replication in a host cell, comprising administering to said host cell an effective amount of a polypeptide inhibitor according to claim 1.

9. The method according to claim 8, wherein inhibiting HIV-1 transcription and replication in a host cell is performed in vitro.

10. The method according to claim 8, wherein inhibiting HIV-1 transcription and replication in a host cell is performed in vivo.

11. The method according to claim 8, wherein said effective amount is between about 100 nM and about 100 μM.

12. A composition for inhibiting HIV-1 transcription and replication in a host cell, the composition comprising the polypeptide inhibitor according to claim 1 and a compatible vehicle.

13. A method of treating or preventing a condition associated with presence in an individual of HIV-1, the method comprising administering to the individual a therapeutic effective amount of the polypeptide inhibitor according to claim 1.

14. The method according to claim 13, wherein said effective amount is between about 10 nM and about 1 mM

15. The method according to claim 13, wherein said effective amount is between about 100 nM and about 100 μM

16. A pharmaceutical composition for inhibiting HIV-1 transcription and replication comprising the polypeptide inhibitor according to claim 1 and a pharmaceutically acceptable vehicle.

17. The pharmaceutical composition according to claim 16, wherein said composition is formulated for parenteral or systemic administration.

18. The pharmaceutical composition according to claim 16, wherein said composition is in form of an injectable solution, an injectable suspension, a tablet or a capsule.

19. A method for producing a polypeptide inhibitor of HIV-1 transcription and replication, the method comprising:
selecting a nuclear localization signal of IκB-α, or a derivative thereof, thus obtaining a selected nuclear localization signal;
selecting a C-terminal nuclear export signal of IκB-α, or a derivative thereof, thus obtaining a selected nuclear export signal;
selecting a binding site of IκB-α for an HIV-1 Tat transactivator, or a derivative thereof, thus obtaining a selected Tat binding site; and
forming said polypeptide inhibitor of HIV-1 transcription and replication with the selected nuclear localization signal, the selected nuclear export signal and the selected Tat binding site.

20. A polypeptide inhibitor of HIV-1 transcription and replication, the polypeptide comprising
an amino acid sequence consisting of SEQ ID NO:12, or a derivative thereof,
an amino acid sequence consisting of SEQ ID NO:13, or a derivative thereof, and/or
an amino acid sequence consisting of SEQ ID NO:14, or a derivative thereof.


SEQUENCE LISTING (14 sequences)

SEQ ID NO: 1 - protein, 216 aa, Artificial Sequence
  Sequence from IkB-alpha protein of Homo Sapiens
EDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEE

SEQ ID NO: 2 - DNA, 55 bp, artificial
  primer
atacagcagcagctgggccaggcgacagcagaaaacgcgcagatgctgccagaga

SEQ ID NO: 3 - DNA, 39 bp, artificial
  primer
ctggcccagctgctgctgtatccgggtgcttgggcggcc

SEQ ID NO: 4 - DNA, 39 bp, artificial
  primer
aaggagctgcaggaggcgcgcgcggagccgcaggaggtg

SEQ ID NO: 5 - DNA, 29 bp, artificial
  primer
ctcctgcagctccttgaccatggagtcca

SEQ ID NO: 6 - DNA, 46 bp, artificial
  primer
cgagcttgctacaagggatctagatccagggaggcgtggcctgggc

SEQ ID NO: 7 - DNA, 39 bp, artificial
  primer
tccttgtagcaagctcgatgtcagcagttcttgaagtac

SEQ ID NO: 8 - DNA, 20 bp, artificial
  primer
tgtgtgcccgtctgttgtgt

SEQ ID NO: 9 - DNA, 20 bp, artificial
  primer
gagtcctgcgtcgagagagc

SEQ ID NO: 10 - DNA, 19 bp, artificial
  primer
gaaggtgaaggtcggagtc

SEQ ID NO: 11 - DNA, 20 bp, artificial
  primer
gaagatggtgatgggatttc

SEQ ID NO: 12 - protein, 11 aa, Artificial Sequence
  Sequence from IkB-alpha protein of Homo Sapiens
LQQTPLHLAVI

SEQ ID NO: 13 - protein, 13 aa, Artificial Sequence
  Sequence from IkB-alpha protein of Homo Sapiens
IQQQLGQLTLENL

SEQ ID NO: 14 - protein, 7 aa, Artificial Sequence
  Sequence from IkB-alpha protein of Homo Sapiens
TRIQQQL